(19) United States
(12) Patent Application Publication
(10) Pub. No.: US 2014/0081406 A1
(43) Pub. Date: Mar. 20, 2014

(54) PROSTHETIC JOINT WITH ALIGNMENT FEATURE

(71) Applicant: Biomedflex, LLC, Gastonia, NC (US)

(72) Inventors: Franz W. Kellar, Gastonia, NC (US); Harold Lloyd Crowder, Jr., Concord, NC (US); Scott M. Duquette, Clemmons, NC (US)

(73) Assignee: BIOMEDFLEX, LLC, Gastonia, NC (US)

(21) Appl. No.: 14/087,581

(22) Filed: Nov. 22, 2013

Related U.S. Application Data
(63) Continuation-in-part of application No. 14/060,080, filed on Oct. 22, 2013.
Application No. 14/060,080 is a continuation of application No. 13/046,311, filed on Mar. 11, 2011.
Application No. 13/046,311 is a continuation-in-part of application No. 12/714,288, filed on Feb. 26, 2010, now Pat. No. 7,905,919.
Application No. 12/714,288 is a continuation-in-part of application No. 11/936,601, filed on Nov. 7, 2007.
Application No. 13/046,311 is a continuation-in-part of application No. 12/826,620, filed on Jun. 29, 2010, now Pat. No. 7,914,580.
Application No. 12/826,620 is a continuation-in-part of application No. 12/714,288, filed on Feb. 26, 2010, now Pat. No. 7,905,919.
Application No. 12/714,288 is a continuation-in-part of application No. 11/936,601, filed on Nov. 7, 2007.
(60) Provisional application No. 60/864,667, filed on Nov. 7, 2006.
(60) Provisional application No. 60/864,667, filed on Nov. 7, 2006.

(51) Int. Cl. A61F 2/30

(52) U.S. Cl. CPC A61F 2/30
USPC 623/18.11

(57) ABSTRACT

A prosthetic joint includes: (a) a first member including a bone-implantable surface, comprising rigid material, and including a body having a cantilevered perimeter flange that defines a wear-resistant, concave first contact surface having a protruding rim and a recessed central portion; (b) a second member including a bone-implantable surface and comprising rigid material with a wear-resistant, convex second contact surface; (c) where the contact surfaces bear directly against each other, transferring axial and lateral loads between the members, while allowing pivoting motion therebetween; (d) wherein the flange is shaped and sized so as to deform elastically, permitting the first contact surface to conform in an irregular shape to the second contact surface when the joint is loaded; and (e) wherein at least one of the bone-implantable surfaces includes at least one mechanical feature configured to maintain the associated member in a specific position and/or orientation relative to bone.

### CROSS-REFERENCE TO RELATED APPLICATIONS

This application is a Continuation-in-Part of application Ser. No. 14/060,080, filed Oct. 22, 2013, which is currently pending, which is a Continuation of application Ser. No. 13/046,311, filed Mar. 11, 2011, which is currently pending, which is a Continuation-in-Part of application Ser. No. 12/714,288, filed Feb. 26, 2010, now U.S. Pat. No. 7,905,919, which is a Continuation-in-Part of application Ser. No. 11/936,601, filed Nov. 7, 2007, which is currently pending, which claims the benefit of Provisional Application 60/864,667 filed Nov. 7, 2006. Application Ser. No. 13/046,311 is also a Continuation-in-Part of application Ser. No. 12/826,620, filed Jun. 29, 2010, now U.S. Pat. No. 7,914,580, which is a Continuation-in-Part of application Ser. No. 12/714,288, filed Feb. 26, 2010, now U.S. Pat. No. 7,905,919, and which is a Continuation-in-Part of application Ser. No. 11/936,601, filed Nov. 7, 2007, which is currently pending, which claims the benefit of Provisional Application 60/864,667 filed Nov. 7, 2006.

### BACKGROUND OF THE INVENTION

This invention relates generally to medical implants, and more particularly to prosthetic joints having conformal geometries and wear resistant properties.

Medical implants, such as knee, hip, and spine orthopedic replacement joints and other joints and implants have previously consisted primarily of a hard metal motion element that engages a polymer contact pad. This has usually been a high density high wear resistant polymer, for example Ultra-High Molecular Weight Polyethylene (UHMWPE), or other resilient material. The problem with this type of configuration is the polymer eventually begins to degrade due to the caustic nature of blood, the high impact load, and high number of load cycles. As the resilient member degrades, pieces of polymer may be liberated into the joint area, often causing accelerated wear, implant damage, and tissue inflammation and harm.

It is desirable to employ a design using a hard member on a hard member (e.g. metals or ceramics), thus eliminating the polymer. Such a design is expected to have a longer service life. Extended implant life is important as it is now often required to revise or replace implants. Implant replacement is undesirable from a cost, inconvenience, patient health, and resource consumption standpoint.

Implants using two hard elements of conventional design will be, however, subject to rapid wear. First, a joint having one hard, rigid element on another will not be perfectly shaped to a nominal geometry. Such imperfections will result in points of high stress, thus causing localized wear. Furthermore, two hard elements would lack the resilient nature of a natural joint. Natural cartilage has a definite resilient property, absorbing shock and distributing periodic elevated loads. This in turn extends the life of a natural joint and reduces stress on neighboring support bone and tissue. If two rigid members are used, this ability to absorb the shock of an active lifestyle could be diminished. The rigid members would transmit the excessive shock to the implant to bone interface. Some cyclical load in these areas stimulates bone growth and strength; however, excessive loads or shock stress or impulse loading the bone-to-implant interface will result in localized bone mass loss, inflammation, and reduced support.

### BRIEF SUMMARY OF THE INVENTION

These and other shortcomings of the prior art are addressed by the present invention, which provides a prosthetic joint having wear-resistant contacting surfaces with conformal properties.

According to one aspect of the invention, a prosthetic joint includes: (a) a first member including a bone-implantable surface, the first member comprising a rigid material and including a body having a cantilevered perimeter flange extending therefrom, the flange defining a wear-resistant, concave first contact surface having a protruding rim and a recessed central portion; and (b) a second member including a bone-implantable surface, the second member comprising a rigid material with a wear-resistant, convex second contact surface; (c) where the first and second contact surfaces bear directly against each other so as to transfer axial and lateral loads from one of the members to the other, while allowing pivoting motion between the two members; (d) wherein the flange is shaped and sized so as to deform elastically and permit the first contact surface to conform in an irregular shape to the second contact surface when the joint is placed under a predetermined load; and (e) wherein at least one of the bone-implantable surfaces includes at least one mechanical feature configured to maintain the associated member in a specific position and/or orientation relative to bone, either during or after implantation.

### BRIEF DESCRIPTION OF THE DRAWINGS

The invention may be best understood by reference to the following description taken in conjunction with the accompanying drawing figures in which:

FIG. 1 is a cross-sectional view of a portion of a resilient contact member constructed in accordance with the present invention;

FIG. 2 is an enlarged view of the contact member of FIG. 1 in contact with a mating joint member;

FIG. 3 is a side view of a resilient contact member in contact with a mating joint member;

FIG. 4 is a cross-sectional view of a cup for an implant according to an alternate embodiment of the invention;

FIG. 5 is an enlarged view of a portion of the cup of FIG. 4;

FIG. 6 is a perspective view of a finite element model of a joint member;

FIG. 7 is a cross-sectional view of an implant joint including a flexible seal;

FIG. 8 is an enlarged view of a portion of FIG. 7;

FIG. 9 is a side view of a prosthetic joint constructed in accordance with an aspect of the present invention;

FIG. 10 is a cross-sectional view of the prosthetic joint of FIG. 9 in an unloaded condition;

FIG. 11 is a cross-sectional view of one of the members of the prosthetic joint of FIG. 9;

FIG. 12 is an enlarged view of a portion of FIG. 10;

FIG. 13 is a cross-sectional view of the prosthetic joint of FIG. 9 in a loaded condition;

FIG. 14 is an enlarged view of a portion of FIG. 13;

FIG. 15 is a cross-sectional view of an alternative joint member;

FIG. 16 is an enlarged view of a portion of FIG. 15;

FIG. 17 is a cross-sectional view of another alternative joint member;

FIG. 18 is a cross-sectional view of another alternative joint member including a filler material;

FIG. 19 is a cross-sectional view of another alternative joint member including a wiper seal;

FIG. 20 is a cross-sectional view of another alternative prosthetic joint;

FIG. 21 is a cross-sectional view of a prosthetic joint constructed in accordance with another aspect of the present invention;

FIG. 22 is a cross-sectional view of a prosthetic joint constructed in accordance with yet another aspect of the present invention; and

FIG. 23 is a perspective view of a joint member having a grooved surface.

FIG. 24 is a exploded perspective view of two mating joint members;

FIG. 25 is a top plan view of one of the joint members shown in FIG. 24;

FIG. 26 is a cross-sectional view of one of the joint members shown in FIG. 24;

FIG. 27 is a contact stress plot of the joint member shown in FIG. 26;

FIG. 28 is a perspective view of a rigid joint member used for comparison purposes;

FIG. 29 is a cross-sectional view of the joint member shown in FIG. 28;

FIG. 30 is a contact stress plot of the joint member shown in FIG. 29;

FIG. 31 is an enlarged view of a portion of the joint shown in FIG. 15;

FIG. 32 is a graph showing contact pressure of the joint of FIG. 15 compared to the number of operating cycles;

FIG. 33 is a greatly enlarged cross-sectional view of a portion of the joint shown in FIG. 31 in an initial condition;

FIG. 34 is a greatly enlarged cross-sectional view of a portion of the joint shown in FIG. 31 after an initial wear-in period;

FIG. 35 is a cross-sectional view of a prosthetic joint constructed in accordance with yet another aspect of the present invention;

FIG. 36 is a plan view of a prosthetic joint member with a saddle-type flange;

FIG. 37 is a view taken along lines 37-37of FIG. 36;

FIG. 38 is a view taken along lines 38-38 of FIG. 36;

FIG. 39 is a perspective view of an alternative prosthetic joint;

FIG. 40 is an exploded view of the prosthetic joint of FIG. 39;

FIG. 41 is an inverted perspective view of a mounting member of the prosthetic joint of FIG. 39;

FIG. 42 is an inverted perspective view of a first bearing member of the prosthetic joint of FIG. 39;

FIG. 43 is a cross-sectional view of a first bearing member of the prosthetic joint of FIG. 39;

FIG. 44 is a perspective view of a second bearing member of the prosthetic joint of FIG. 39;

FIG. 45 is an exploded perspective view of a joint member of the prosthetic joint of FIG. 39;

FIGS. 46 through 49 are schematic cross-sectional views of joint members incorporating various types of alignment elements;

FIG. 50 is a perspective view of a joint member having an instrument-engaging feature formed therein;

FIG. 51 is a top plan view of the joint member of FIG. 50;

FIG. 52 is a top plan view of a portion of a joint member, showing an alternative instrument-engaging feature;

FIG. 53 is a top plan view of a portion of a joint member, showing an alternative instrument-engaging feature;

FIG. 54 is a side elevation view of a joint member including a bracket configured to receive a fastener for securing the joint member to bone;

FIG. 55 is a schematic perspective view of a joint member having a tapered alignment element;

FIG. 56 is a top plan view of the joint member of FIG. 55;

FIG. 57 is a schematic perspective view of a joint member having an interrupted alignment element;

FIG. 58 is a side view of the joint member of FIG. 57;

FIG. 59 is a schematic perspective view of a joint member having an interrupted alignment element;

FIG. 60 is a side view of the joint member of FIG. 59;

FIG. 61 is a schematic perspective view of a joint member having a grooved alignment element; and

FIG. 62 is a side view of the joint member of FIG. 60.

### DETAILED DESCRIPTION OF THE INVENTION

The present invention provides a specialized implant contact interface (implant geometry). In this geometry, an implanted joint includes two typically hard (i.e. metal or ceramic) members; however, at least one of the members is formed such that it has the characteristics of a resilient member, such as: the ability to absorb an impact load; the ability to absorb high cycle loading (high endurance limit); the ability to be self cleaning; and the ability to function as a hydrodynamic and/or hydrostatic bearing.

Generally, the contact resilient member is flexible enough to allow elastic deformation and avoid localized load increases, but not so flexible as to risk plastic deformation, cracking and failure. In particular, the resilient member is designed such that the stress levels therein will be below the high-cycle fatigue endurance limit. As an example, the resilient member might be only about 10% to about 20% as stiff as a comparable solid member. It is also possible to construct the resilient member geometry with a variable stiffness, i.e. having a low effective spring rate for small deflections and a higher rate as the deflections increase, to avoid failure under sudden heavy loads.

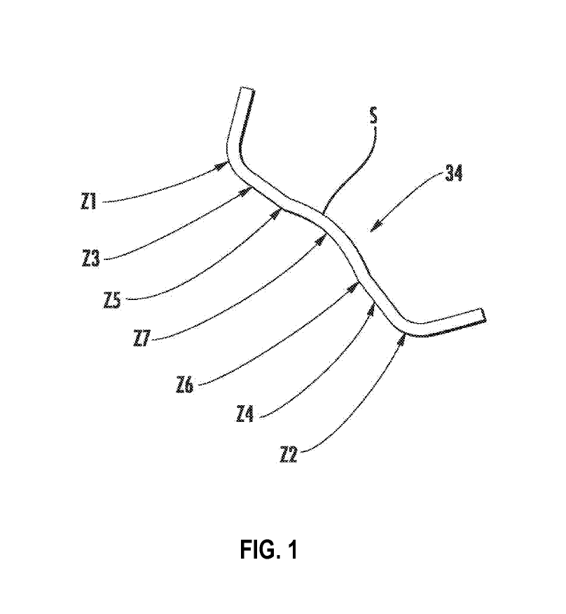

FIG. 1 illustrates an exemplary contact member 34 including a basic resilient interface geometry. The contact member 34 is representative of a portion of a medical implant and is made of one or more metals or ceramics (for example, partially stabilized Zirconia). It may be coated as described below. The geometry includes a lead-in shape, Z1 and Z2, a contact shape, Z3 and Z4, a lead-out shape, Z5 and Z6, and a relieved shape, Z7. It may be desired to vary the cross-sectional thickness to achieve a desired mechanical stiffness to substrate resilience characteristic. The presence of the relieved region Z7 introduces flexibility into the contact member 34, reduces the potential for concentrated point contact with a mating curved member, and provides a reservoir for a working fluid.

The Z7 region may be local to the contact member 34 or may be one of several. In any case, it may contain a means of providing fluid pressure to the internal contact cavity to produce a hydrostatic interface. A passive (powered by the regular motion of the patient) or active (powered by micro components and a dedicated subsystem) pumping means and optional filtration may be employed to provide the desired fluid interaction.

A hydrodynamic interface is desirable as, by definition, it means the contact member 34 is not actually touching the mating joint member. The lead-in and lead-out shapes Z1, Z2, Z5, Z6 are configured to generate a shear stress in the working fluid so as to create the fluid “wedge” of a hydrodynamic support.

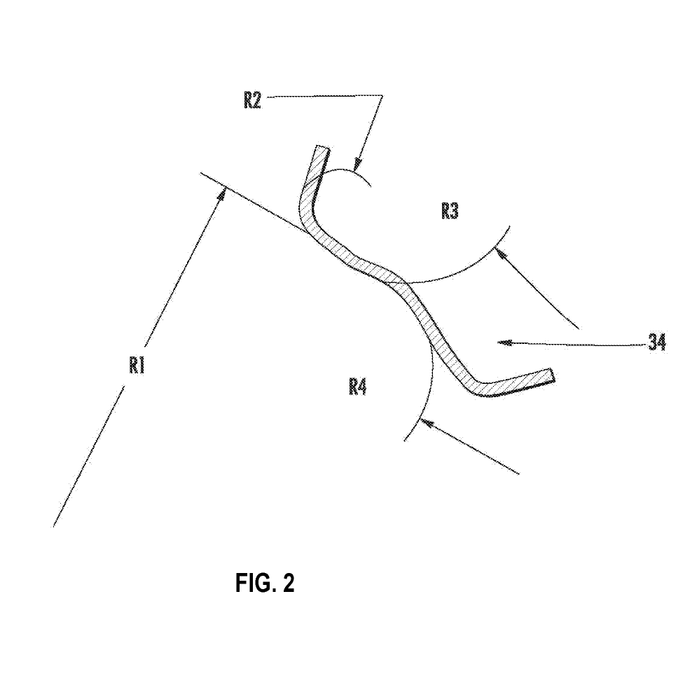

FIG. 2 shows a closer view of the contact member 34, with its various defining curves labeled R1-R4. It may be desirable to make the radius of the contact shape (Z3 and Z4) larger or smaller, depending on the application requirement and flexural requirement. For example, FIG. 3 illustrates the contact member 34 in contact with a mating joint member 38 having a substantially larger radius than the contact member 34. The radius ratio between the two joint members is not particularly critical, so long as one of the members exhibits the resilient properties described herein.

The contact member 34 includes an osseointegration surface “S” (see FIG. 1), which is a surface designed to be infiltrated by bone growth to improve the connection between the implant and the bone. Osseointegration surfaces may be made from materials such as TRABECULAR METAL, textured metal, or sintered or extruded implant integration textures. TRABECULAR METAL is an open metal structure with a high porosity (e.g. about 80%) and is available from Zimmer, Inc., Warsaw, Ind. 46580 USA.

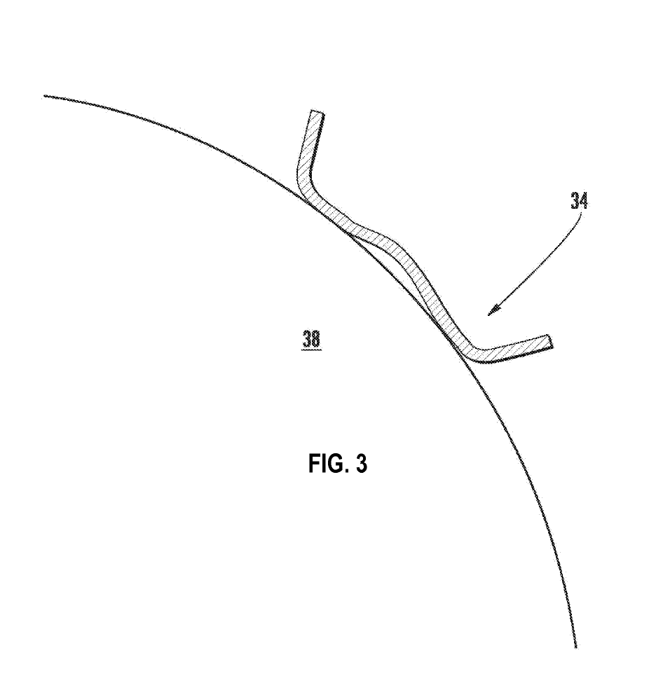

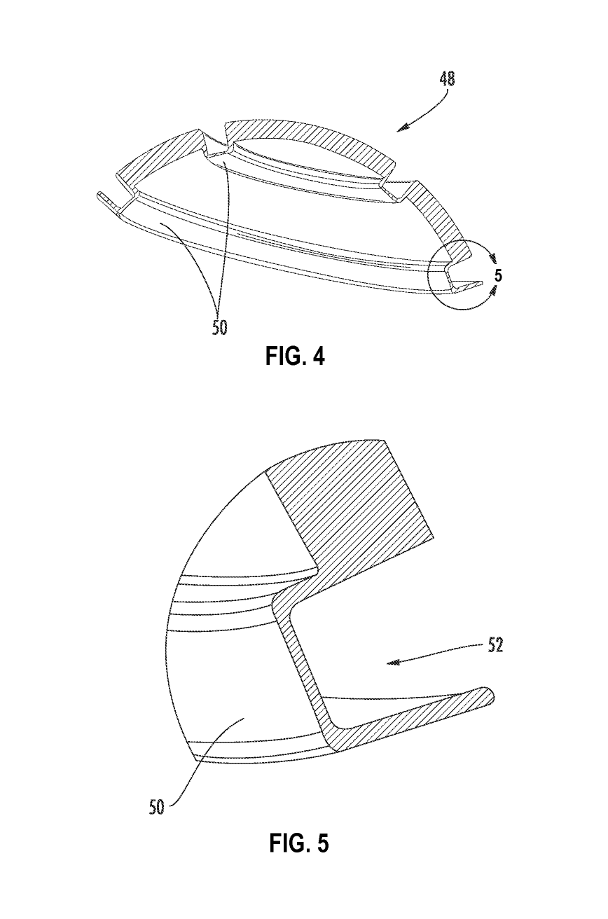

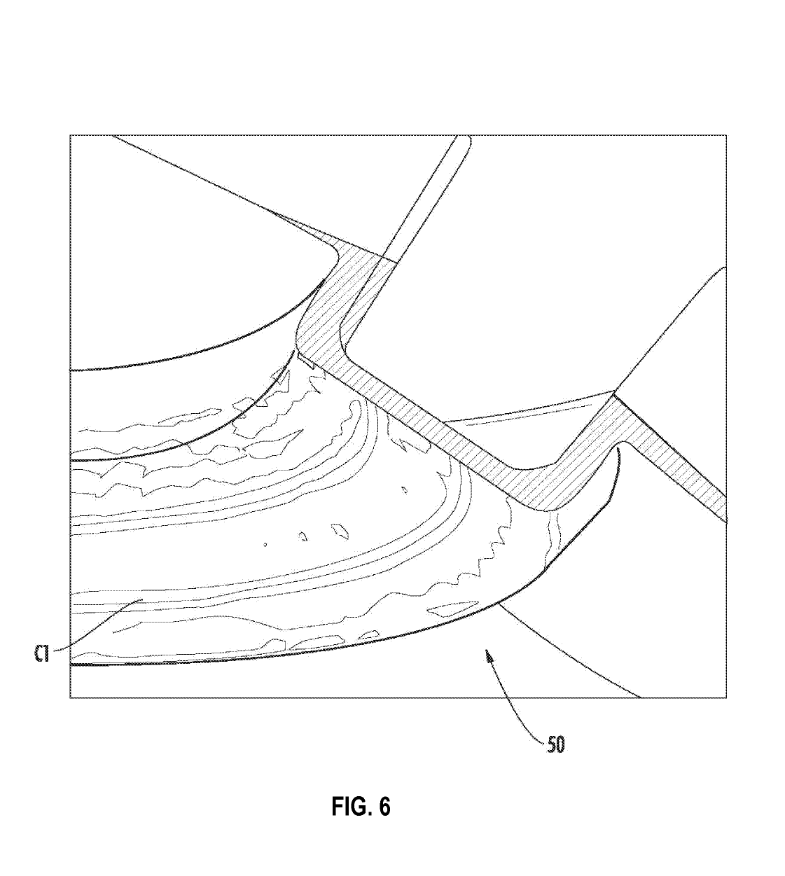

FIGS. 4 and 5 illustrate a cup 48 of metal or ceramic with two integrally-formed contact rings 50. More contact rings may be added if needed. As shown in FIG. 5, the volume behind the contact rings 50 may be relieved. This relieved area 52 may be shaped so as to produce a desired balance between resilience and stiffness. A varying cross-section geometry defined by varying inner and outer spline shapes may be desired. In other words, a constant thickness is not required. A material such as a gel or non-Newtonian fluid (not shown) may be disposed in the relieved area 52 to modify the stiffness and damping characteristics of the contact rings 50 as needed for a particular application. The cup 48 could be used as a stand-alone portion of a joint, or it could be positioned as a liner within a conventional liner. The contact ring 50 is shown under load in FIG. 6, which depicts contour lines of highest compressive stress at “C1”. This is the portion of the contact ring 50 that would be expected to undergo bending first. The bearing interface portion of the resilient contact member could be constructed as a bridge cross-section supported on both sides as shown or as a cantilevered cross-section depending on the desired static and dynamic characteristics.

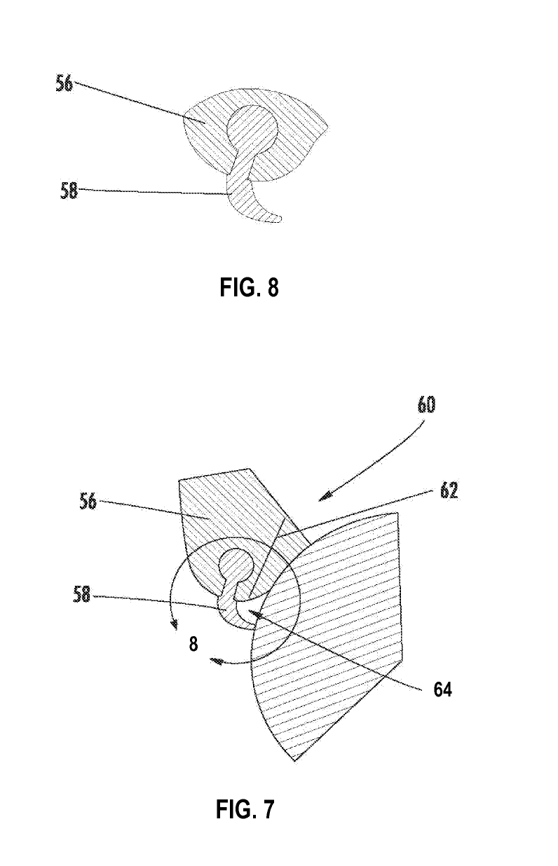

FIGS. 7 and 8 illustrate an implant 56 of rigid material which includes a wiper seal 58. The wiper seal 58 keeps particles out of the contact area (seal void) 60 of the implant 58, and working fluid (natural or synthetic) in. The seal geometry is intended to be representative and a variety of seal characteristics may be employed; such as a single lip seal, a double or multiple lip seal, a pad or wiper seal made from a variety of material options. Different seal mounting options may be used, for example a lobe in a shaped groove as shown in FIGS. 7 and 8, a retaining ring or clamp, or an adhesive. The wiper seal 58 may also be integrated into the contact face of the interface zone.

It may be desirable to create a return passage 62 from the seal void region 60 back into the internal zone 64 in order to stabilize the pressure between the two and to allow for retention of the internal zone fluid if desired. This is especially relevant when the hydrostatic configuration is considered.

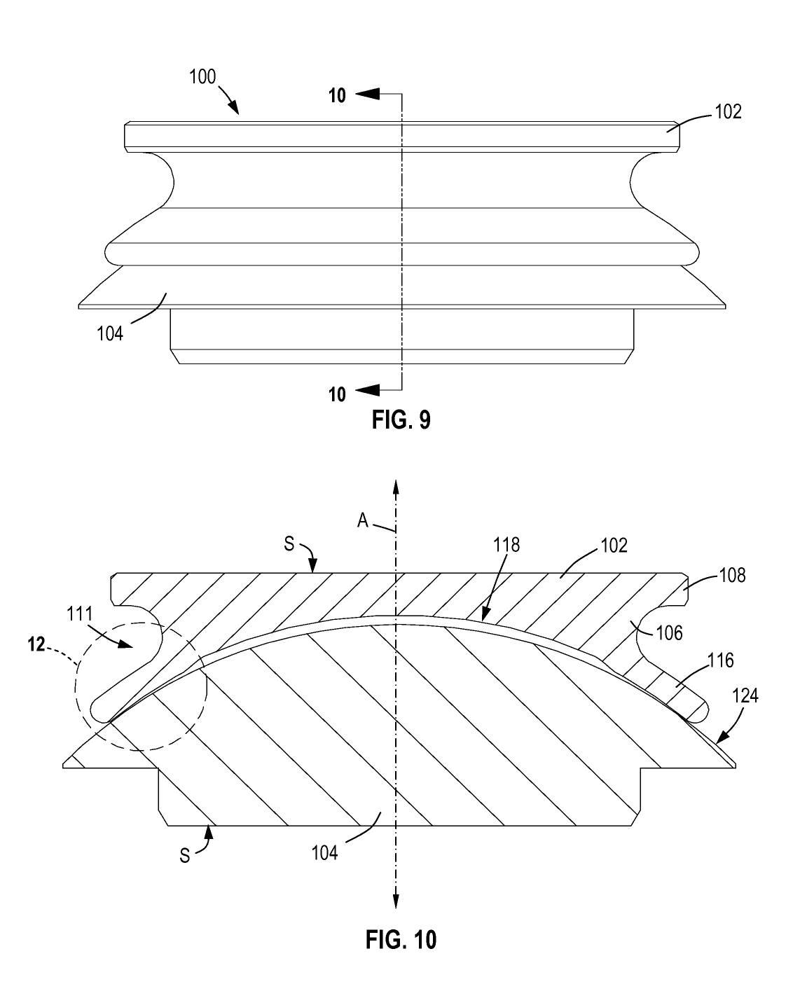

FIGS. 9-14 illustrate a prosthetic joint 100 comprising first and second members 102 and 104. The illustrated prosthetic joint 100 is particularly adapted for a spinal application, but it will be understood that the principles described herein may be applied to any type of prosthetic joint. Both of the members 102 and 104 may be bone-implantable, meaning they include osseointegration surfaces, labeled “S”, which are surfaces designed to be infiltrated by bone growth to improve the connection between the implant and the bone. Osseointegration surfaces may be made from materials such as TRABECULAR METAL, textured metal, or sintered or extruded implant integration textures, as described above. As shown in FIG. 10, a central axis “A” passes through the centers of the first and second members 102 and 104 and is generally representative of the direction in which external loads are applied to the joint 100 in use. In the illustrated examples, the first and second joint members are bodies of revolution about this axis, but the principles of the present invention also extend to shapes that are not bodies of revolution.

The first member 102 includes a body 106 with a perimeter flange 116 extending in a generally radially outward direction at one end. Without regard to the exact direction that the flange 116 extends, a defining feature of the flange is that it is cantilevered relative to the body 106. In other words, when viewed in cross-section, it is a projecting structure, that is supported at one end and carries a load at the other end or along its length. The flange 116 may be open or closed perimeter, and may have varying shapes in plan view (e.g. circular, elliptical, a spline, or an asymmetrical shape). Optionally, a disk-like base 108 may be disposed at the end of the body 106 opposite the flange 116, in which case a circumferential gap 111 will be defined between the base 106 and the flange 116. The first member 102 is constructed from a rigid material. As used here, the term “rigid” refers to a material which has a high stiffness or modulus of elasticity. Nonlimiting examples of rigid materials having appropriate stiffness for the purpose of the present invention include stainless steels, cobalt-chrome alloys, titanium, aluminum, and ceramics. By way of further example, materials such as polymers would generally not be considered “rigid” for the purposes of the present invention. Generally, a rigid material should have a modulus of elasticity of about 0.5×10⁶ psi or greater. Collectively, one end of the body 106 and the flange 116 define a wear-resistant, concave first contact surface 118. As used herein, the term “wear-resistant” refers to a material which is resistant to surface material loss when placed under load. Generally the wear rate should be no more than about 0.5 μm (0.000020 in.) to about 1.0 μm (0.000040 in.) per million cycles when tested in accordance with ASTM Guide F2423. As a point of reference, it is noted that any of the natural joints in a human body can easily experience one million operating cycles per year. Nonlimiting examples of wear-resistant materials include solid metals and ceramics. Known coatings such as titanium nitride, chrome plating, carbon thin films, and/or diamond-like carbon coatings may be used as a face layer to impart wear resistance to the first contact surface 118. Optionally, the first contact surface 118 could comprise a substantially thicker face layer (not shown) of a wear-resistant material such as ultra-high molecular weight (UHMW) polyethylene.

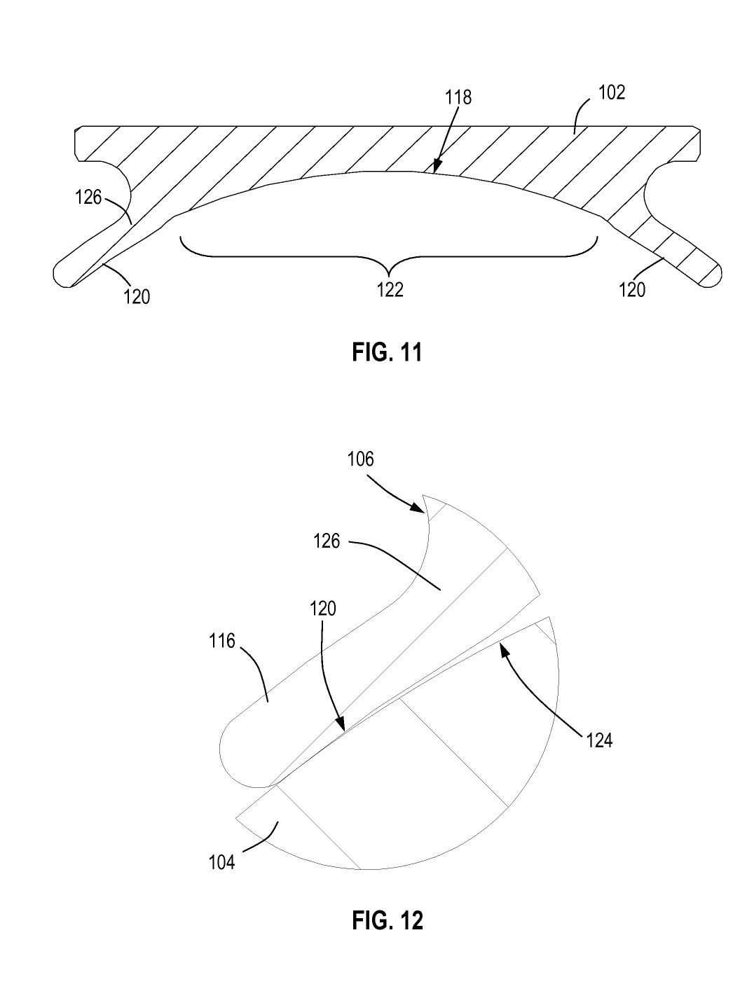

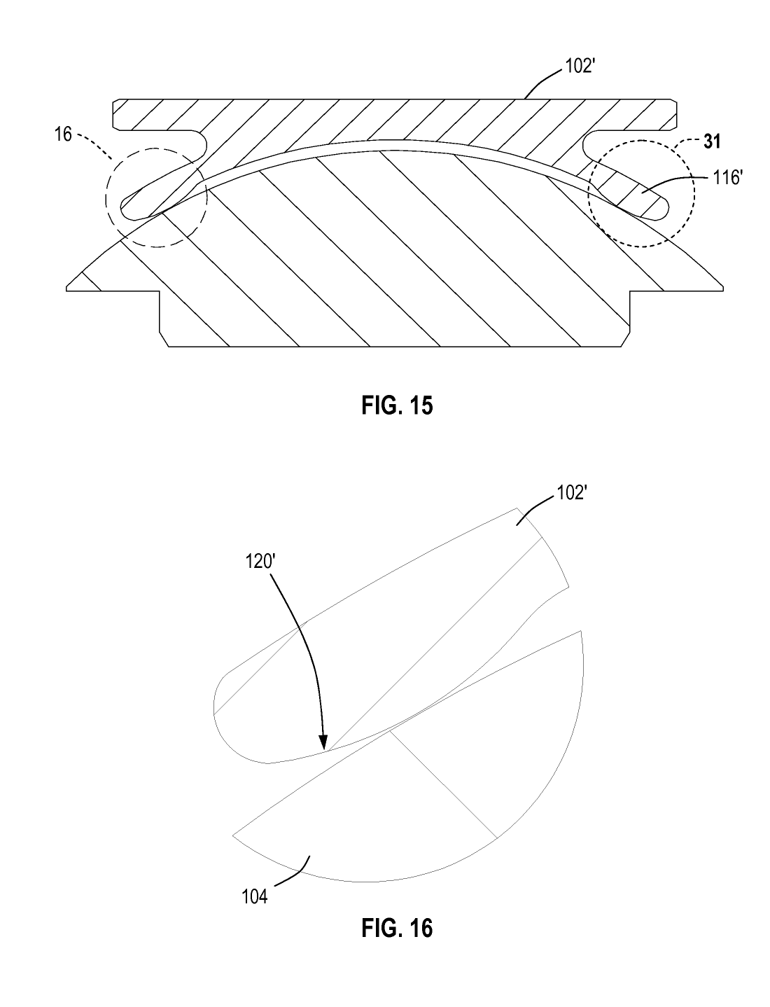

The first contact surface 118 includes a protruding peripheral rim 120 (see FIG. 11), and a recessed central portion 122, which may also be considered a “pocket” or a “relief”. As used herein, the term “recessed” as applied to the central portion 122 means that the central portion 122 lies outside of the nominal exterior surface of the second member 104 when the joint 100 is assembled. The terms “recessed” and “protruding” as used herein are opposite in meaning to one another. For example, the peripheral rim 120 protrudes relative to a nominal surface defined by the central portion 122, and the central portion 122 is recessed relative to the rim 120. In one configuration, shown in FIGS. 9-14, and best seen in FIG. 11, the rim 120 is concave, with the radius of curvature being quite high, such that the cross-sectional shape of the surface of the rim 120 approaches a straight line. FIGS. 15 and 16 show another configuration of a joint member 102′ with a flange 116′, in which the rim 120′ has a convex-curved cross-sectional shape. The cross-sectional shape of the rim may be flat or curved as necessary to suit a particular application.

The second member 104 is also made from a rigid material and has a wear-resistant, convex second contact surface 124. The first and second contact surfaces 118 and 124 bear directly against each other so as to transfer axial and lateral loads from one member to the other while allowing pivoting motion between the two members 102 and 104.

The annular configuration of first contact surface 118 with the protruding rim 120 results in a configuration which permits only pivoting and rotational motion, and is statically and dynamically determinate for the life of the joint 100. In contrast, prior art designs employing mating spherical shapes, even very accurate shapes, quickly reach a statically and dynamically indeterminate condition after use and wear. This condition accelerates wear, contributes to the fretting corrosion wear mechanism, and permits undesired lateral translation between the joint members.

Nominally the first and second members 102 and 104 define a “ring” or “band” contact interface therebetween. In practice it is impossible to achieve surface profiles completely free of minor imperfections and variations. If the first and second members 102 and 104 were both completely rigid, this would cause high Hertzian contact stresses and rapid wear. Accordingly, an important feature of the illustrated joint 100 is that the flange 116 (and thus the first contact surface 118) of the first member 102 is conformable to the second contact surface 124 when the joint is placed under load.

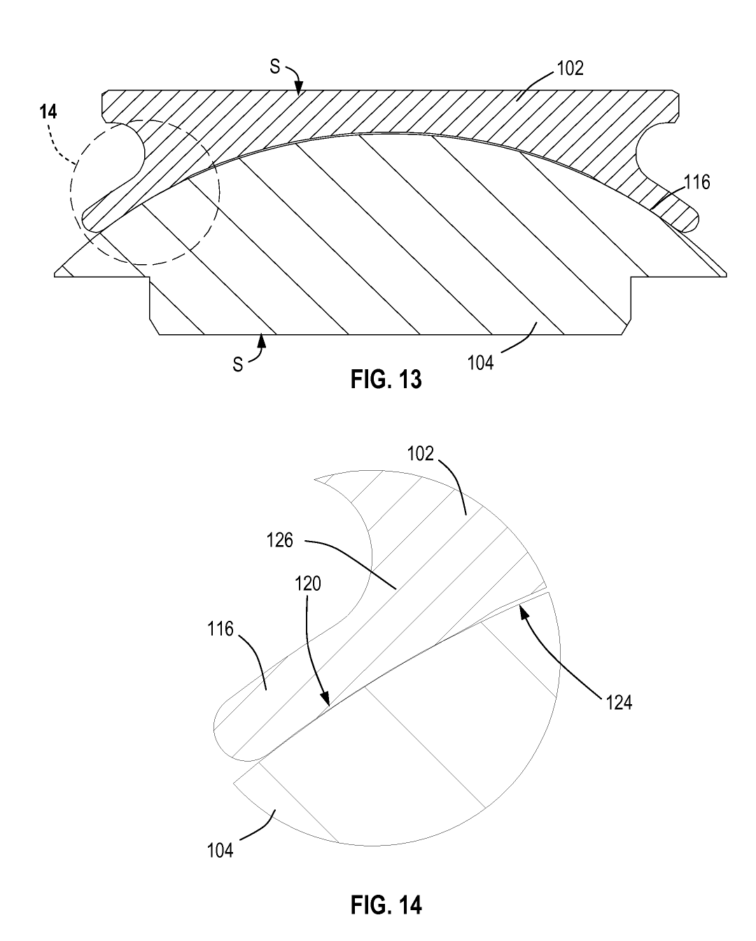

FIGS. 10 and 12 show a cross-sectional view of the flange 116 in an unloaded condition or free shape. It can be seen that the distal end of the rim 120 contacts the second contact surface 124, while the inboard end of the rim 120 (i.e. near where the flange 116 joins the body 106) does not. FIGS. 13 and 14 show the flange 116 in a deflected position or loaded shape, where substantially the entire section width of the rim 120 contacts the second contact surface 124, resulting in a substantially increased contact surface area between the two members 102 and 104, relative to the free shape. The rim 120′ of the joint member 102′ (see FIG. 16) is similarly conformable; however, given the curved cross-sectional shape, the total amount of surface contact area remains substantially constant in both loaded and unloaded conditions, with the rim 120′ undergoing a “rolling” or “rocking” motion as the loading changes.

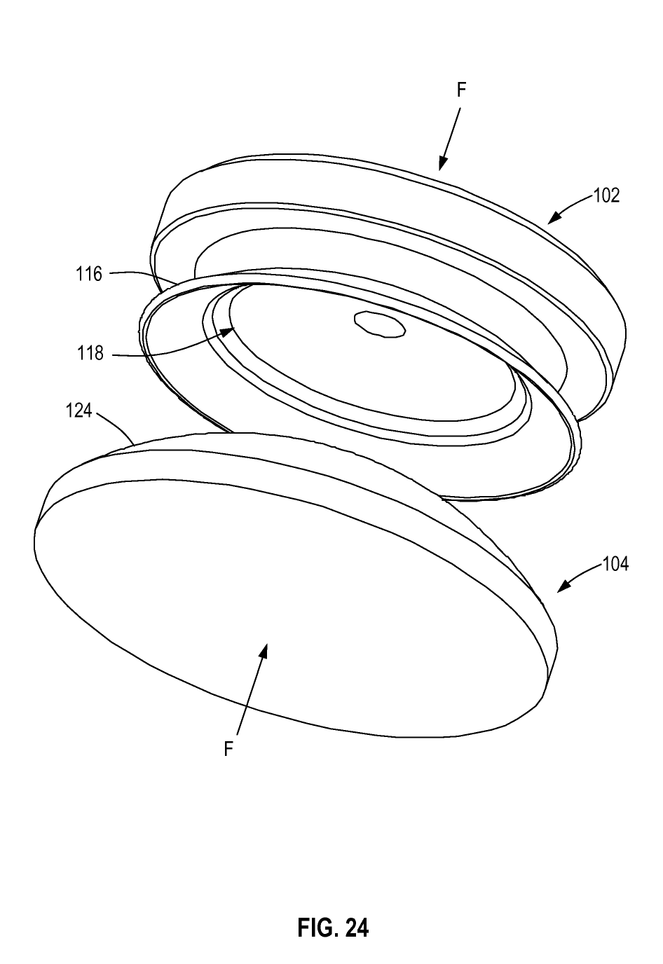

The conformable nature of the flange 116 is explained in more detail with reference to FIGS. 24 through 30. As noted above, the first member 102 has a flange 116 and a concave first contact surface 118. The second member 104 has a convex second contact surface 124. When assembled and in use the joint 100 is subject, among other loads, to axial loading in the direction of the arrows labeled “F” in FIG. 24 (i.e. along axis “A” of FIG. 10). As previously stated, it is impossible in practice for either of the contact surfaces 118 or 124 to be perfect surfaces (i.e. a perfect sphere or other curve or collection of curves). It is believed that in most cases that a defect such as a protrusion from the nominal contact surface of just 0.00127 mm (0.00005 in.), that is, 50 millionths of a inch, or larger, would be sufficient to cause fretting corrosion and failure of a metal-on-metal joint constructed to prior art standards. A defect may include a variance from a nominal surface shape as well as a discontinuity in the contact surface. Defects may arise through a variety of sources such as manufacturing, installation, and/or operating loads in the implanted joint.

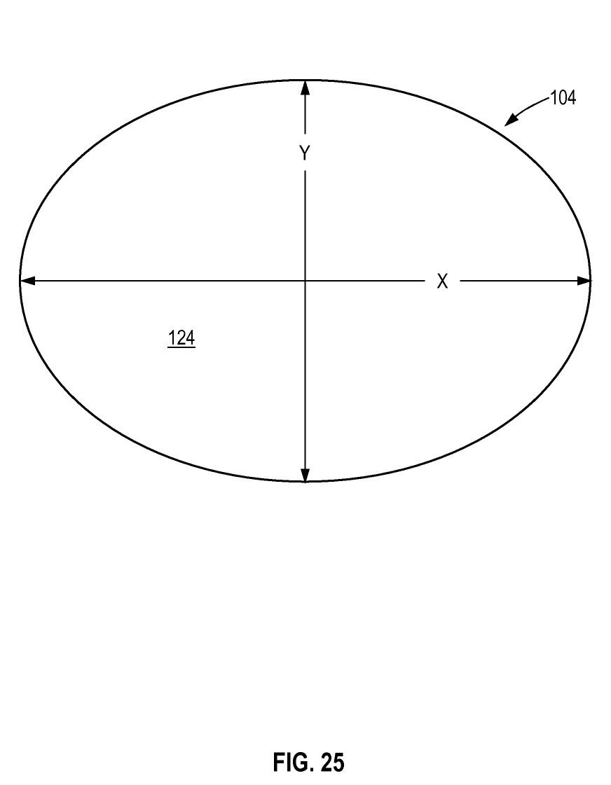

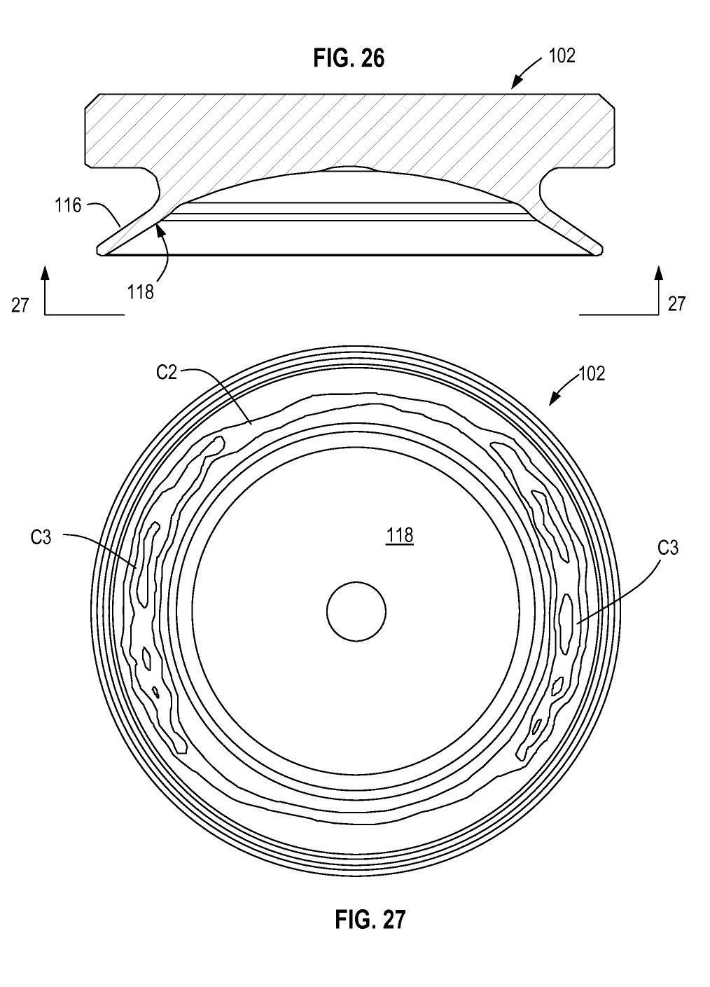

FIG. 25 shows the second member 104 which in this particular example varies from a nominal shape in that it is elliptical rather than circular in plan view. The elliptical shape is grossly exaggerated for illustrative purposes. For reference, the dimensions of the second member 104 along the major axis labeled “X” is about 0.0064 mm (0.00025 in.) larger than its dimension along the minor axis labeled “Y”. When assembled and loaded, the flange 116 conforms to the imperfect second contact surface 124 and deflects in an irregular shape. In other words, in addition to any uniform deflection which may be present, the deflected shape of the flange 116 includes one or more specific locations or portions that are deflected towards or away from the nominal free shape to a greater or lesser degree than the remainder of the flange 116. Most typically the deflected shape would be expected to be non-axisymmetric. For example, the deflection of the flange 116 at points located at approximately the three o'clock and nine o'clock positions is substantially greater than the deflection of the remainder of the flange 116. As a result, the contact stress in that portion of the first contact surface 118 is relieved. FIG. 27 is a plan view plot (the orientation of which is shown by arrow in FIG. 26) which graphically illustrates the expected contact stresses in the first contact surface 118 as determined by analytical methods. The first contour line “C2” shows that a very low level of contract stress is present around the entire perimeter of the first contact surface 118. This is because the entire first contact surface 118 is in contact with the second contact surface 124. Another contour line “C3” represents the areas of maximum contact stress corresponding to the protruding portions of the elliptical second contact surface 124.

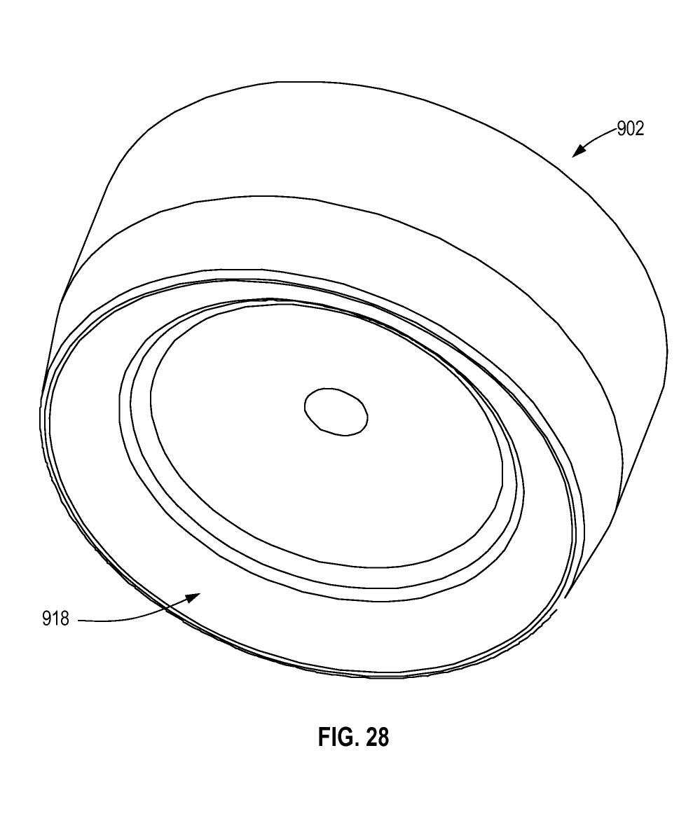

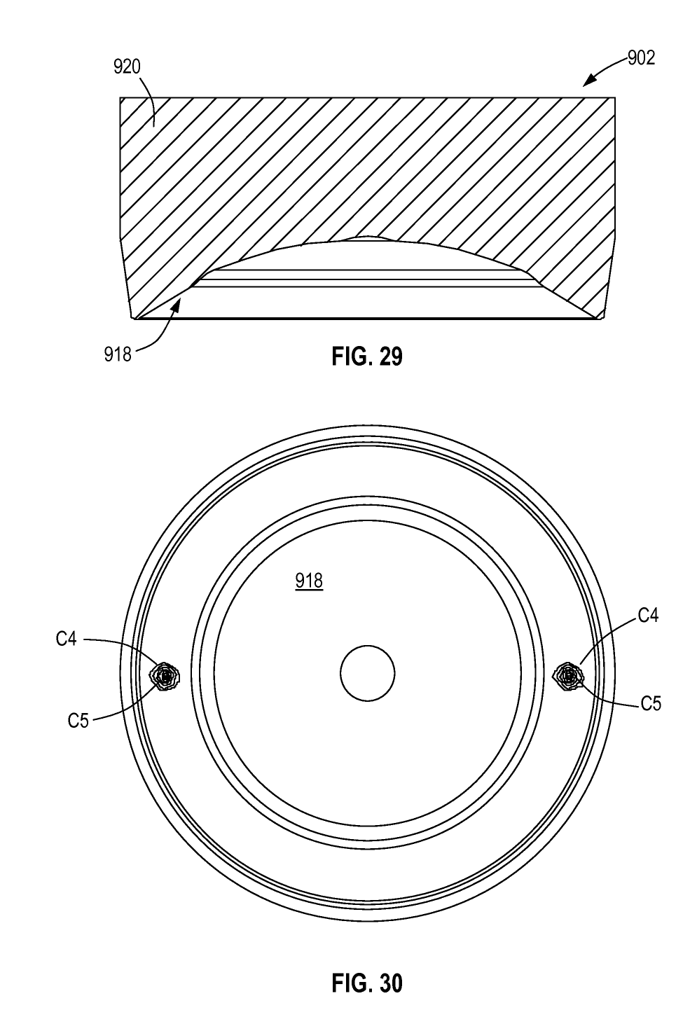

For comparative purposes, FIGS. 28 and 29 depict a member 902 constructed according to prior art principles. The member 902 has a contact surface 918 with an identical profile and dimensions of the first contact surface 118 of the first member 102. However, consistent with the prior art, the member 902 has a massive body 920 behind the entire contact surface 918, rendering the entire member 902 substantially rigid. FIG. 30 graphically illustrates the expected contact stresses in the contact surface 918 as determined by analytical methods, when the member 902 is assembled and placed in contact with the second member 104, using the same applied load as depicted in FIG. 27. Because of the rigidity of the member 902, a “bridging” effect is present wherein contact between the contact surfaces (one of which is circular in plan view, and the other of which is elliptical) effectively occurs at only two points, located at approximately the three o'clock and nine o'clock positions. A first contour line “C4” shows two discrete areas where the lowest level of contract stress is present. These lines are not contiguous because there is no contact in the remaining area of the contact surfaces (for example at the six o'clock and twelve o'clock positions). Another contour line “C5” represents the areas of maximum contact stress. Analysis shows a peak contact stress having a magnitude of two to twenty times (or more) the peak contact stress of the inventive joint as shown in FIG. 27.

To achieve this controlled deflection, the flange 116 is thin enough to permit bending under working loads, but not so thin as to allow material yield or fatigue cracking The deflection is opposed by the elasticity of the flange 116 in bending, as well as the hoop stresses in the flange 116. To achieve long life, the first member 102 is sized so that stresses in the flange 116 will be less than the endurance limit of the material, when a selected external load is applied. In this particular example, the joint 100 is intended for use between two spinal vertebrae, and the design average axial working load is in the range of about 0 N (0 lbs) to about 1300 N (300 lbs.). These design working loads are derived from FDA-referenced ASTM and ISO standards for spinal disc prostheses. In this example, the thickness of the flange 116, at a root 126 where it joins the body 106 (see FIG. 12) is about 0.4 mm (0.015 in.) to about 5.1 mm (0.200 in.), where the outside diameter of the flange 116 is about 6.4 mm (0.25 in.) to about 7.6 cm (3.0 in.).

The rim may be designed in conjunction with the contact surface 124 to create a wear characteristic that is constantly diminishing (similar to an asymptotic characteristic). With reference to FIG. 31, and using the rim 120′ shown in FIG. 15 as an example, the as-manufactured or initial curvature (e.g. radii) of the rim 120′ (labeled “R”) is different from the curvature (e.g. radius) of the contact surface 124 (labeled “r”).

In the illustrated example, the first member 102′ includes a face layer 127 of a known coating such as titanium nitride, chrome plating, carbon thin films, and/or diamond-like carbon coatings, and/or a another wear-resistant material such as ultra-high molecular weight (UHMW) polyethylene. This face layer 127 is used to impart wear resistance, as described above. The face layer 127 may be extraordinarily thin. In this particular example, its as-applied thickness is about 0.0041 mm (0.00016 in.), or 160 millionths of an inch thick. The face layer 127 is applied at a substantially uniform thickness over the surface profile which is defined by machined or formed features of the substrate. Alternatively, and especially if a much thicker face layer were used, the face layer could be profiled so as to define both the nominal cup surface and the rim 120′. The second member 104 may include a similar face layer 130.

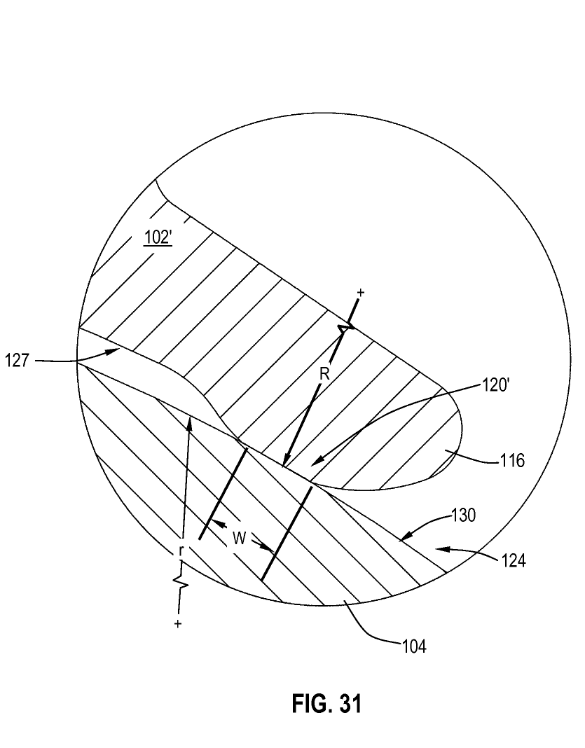

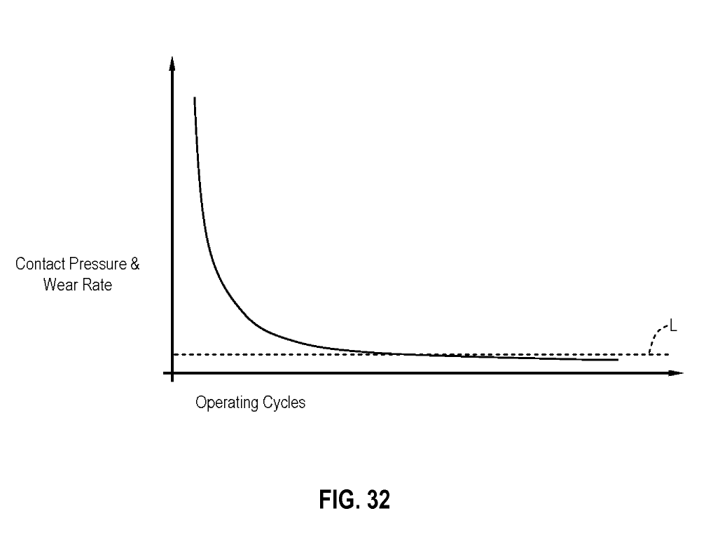

It is noted that the direction of curvature (i.e. the convexity or second derivative shape) of the rim 120′ may be the same as, or opposite to, that of the contact surface 124 upon initial manufacture. In this example they are opposite. When assembled and placed under load, the annular interface between the rim 120′ and the contact surface 124 will have a characteristic width (labeled “W”), effectively creating a contact band. The initial dimensions R and r are selected such that, even using highly wear-resistant surfaces or coatings, some wear takes place during an initial wear-in period of movement cycles. As a result, the contact band width W increases during the initial wear-in period. This increases contact area and therefore decreases contact stress for a given load. After the initial wear-in period (which preferably occurs before the joint is implanted), the contact band reaches a post wear-in width at which the contact stress is below a selected limit, below which the rate of wear in the contacting surfaces approaches a very low number or zero, consistent with a long life of the joint 100. FIG. 32 illustrates this wear characteristic, with the limit “L” depicted as a horizontal line.

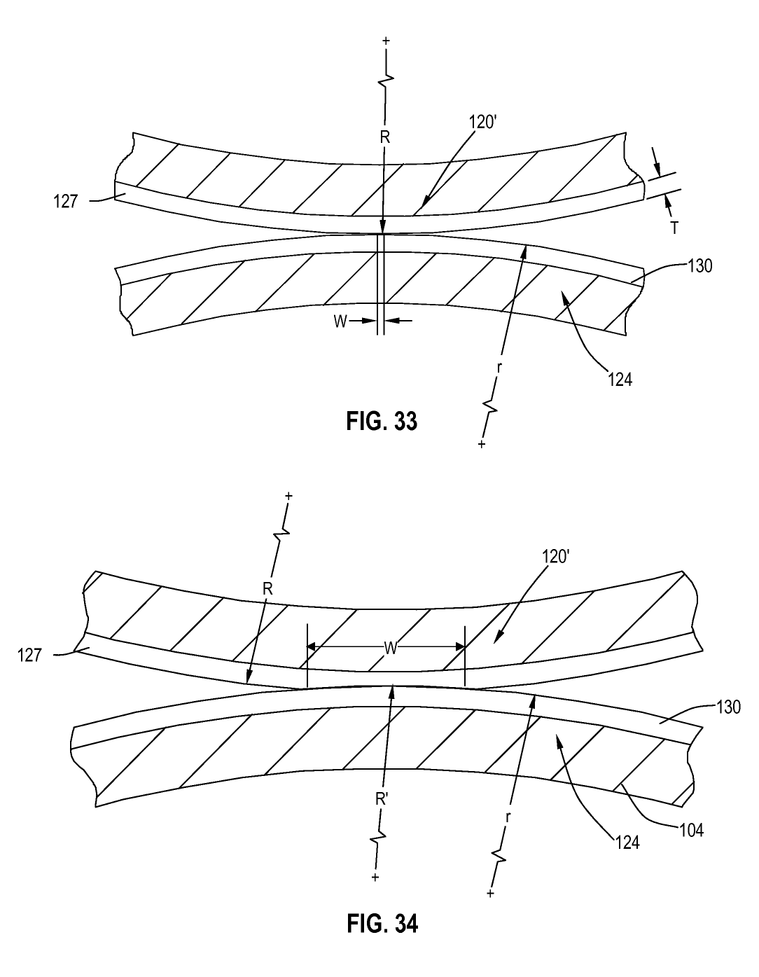

FIGS. 33 and 34 are schematic views showing the initial wear-in of the surface of the rim 120′ at a microscopic (or nearly microscopic) level. It will be understood that these figures are greatly exaggerated for the purposes of illustration. On initial manufacture, as shown in FIG. 33, the curvatures R and r of the rim 120′ and the contact surface 124 have opposite directions.

When assembled, the contact band width W is some nominal value, for example about 0.03 mm (0.001 in.), and the total thickness “T” of the face layer 127 is at its as-applied value of about 0.0041 mm (0.00016 in.) for example. The action of the wear-in period described causes the face layer 127 to wear to a shape complementary to the contact surface 124. After this wear-in period the curvature of the portion of the rim 120′ within the contact band, denoted “R′”, and the curvature r of the contact surface 124 are in the same direction, and the values of the two curvatures are substantially the same. For example, the thickness T at the location of the contact band may decrease by about 0.0004 mm (0.000014 in.), with a corresponding increase in the width of the contact band W to about 0.2 mm (0.008 in.). Analysis shows that this increase in contact band width and surface area can reduce mean contact pressure by over 80%.

The configuration of the flange 116′ is important in developing the constantly diminishing wear characteristics described above. In particular, the flange 116′ is sized and shaped so that deflections of the rim 120′ under varying load is always essentially normal to its tangent points on the opposing contact surface 124, as the joint 100 is loaded and unloaded. This ensures that the position of the contact band remains constant and that the contact band remains substantially uniform around the entire periphery of the joint 100.

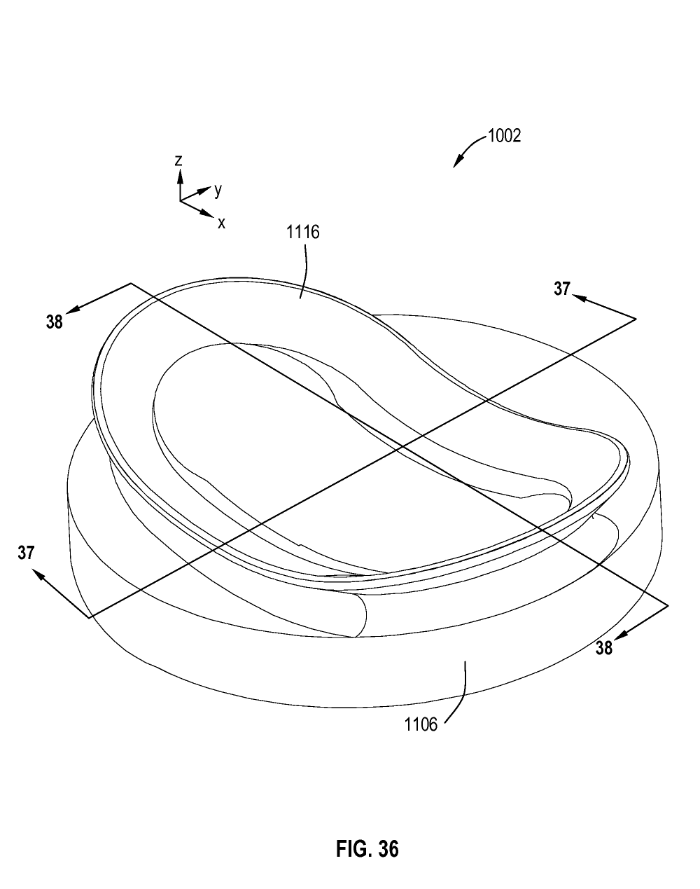

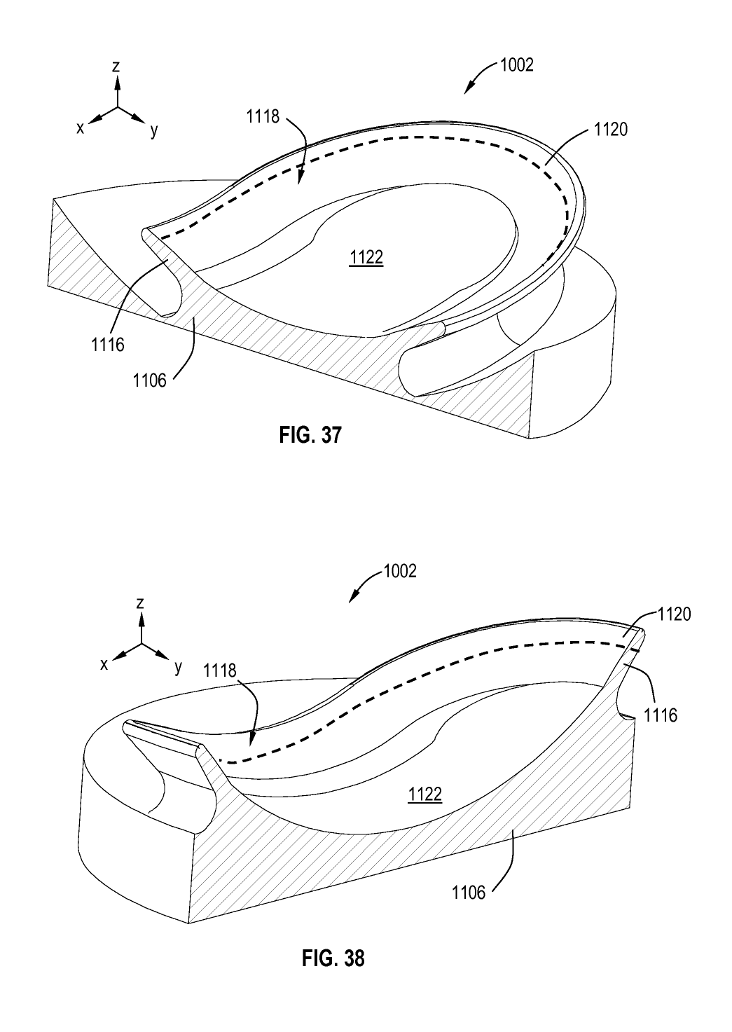

The flange of the joint member need not be circular, elliptical, or another symmetrical shape in plan view, and need not lie in a single plane. For example, FIGS. 36-38 illustrate a joint member 1002 with a body 1106 and a flange 1116 which cooperatively define a contact surface 1118 with a recessed central portion 1122 and a protruding rim 1120. The flange 1116 (and therefore the rim 1120) have a “saddle” shape, meaning that their contours vary in the x-, y-, and z-directions. In this particular example it has a racetrack shape in plan view, and the portions at the ends of the major axis of the racetrack shape are elevated (in the z-direction) relative to the remainder of the shape. The rim 1120 is shaped so as to define a contact band (shown by the heavy line) in which some or all points on its surface lie on a sphere (or otherwise match the shape of the mating joint member).

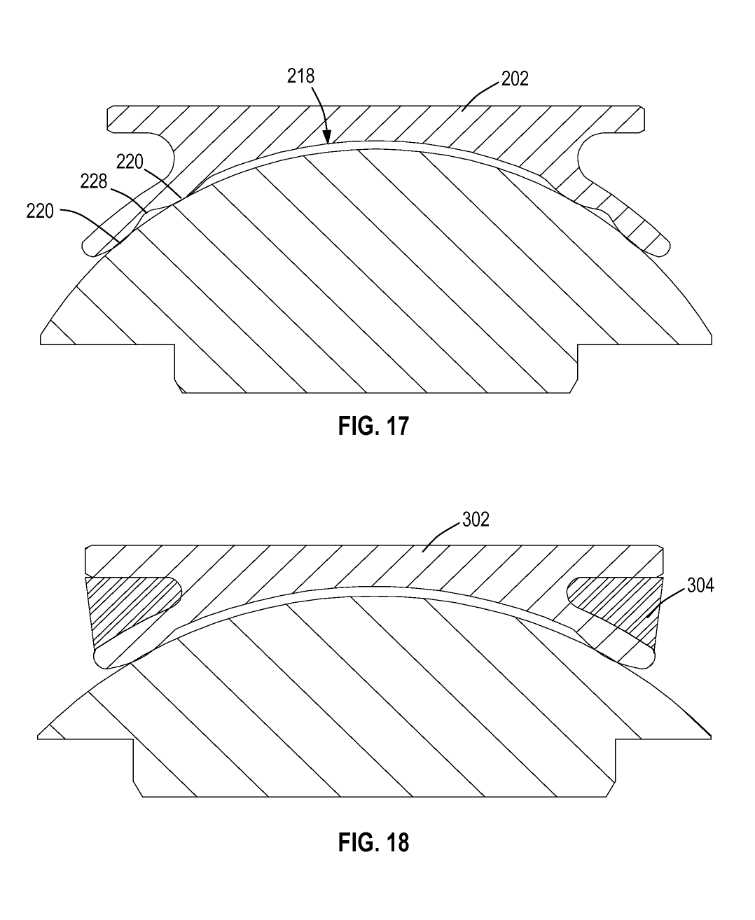

The joint members may include multiple rims. For example, FIG. 17 illustrates a joint member 202 where the first contact surface 218 includes two protruding rims 220, with a circumferential groove or relief area 228 therebetween. The presence of multiple rims increases the contact surface areas between the two joint members.

If present, the circumferential gap between the flange and the base of the joint member may be filled with resilient nonmetallic material to provide damping and/or additional spring restoring force to the flange. FIG. 18 illustrates a joint member 302 with a filler 304 of this type. Examples of suitable resilient materials include polymers, natural or synthetic rubbers, and the like.

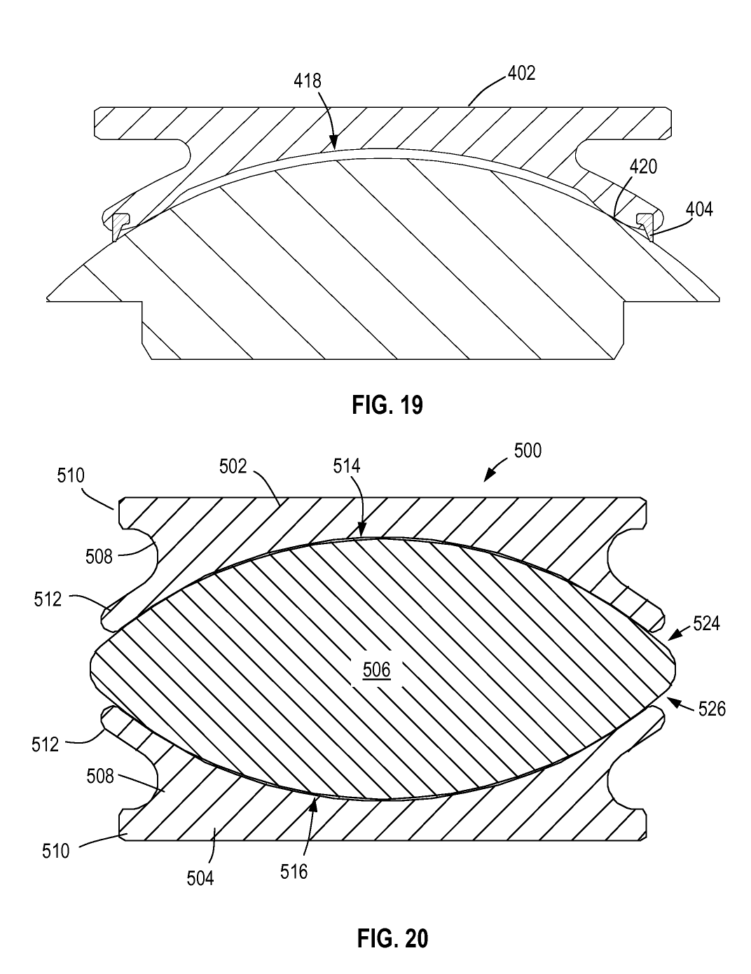

As discussed above, the joint may incorporate a wiper seal. For example, FIG. 19 illustrates a joint member 402 with a resilient wiper seal 404 protruding from the rim 420 of the first contact surface 418. The wiper seal 404 keeps particles out of the contact area (seal void), while containing working fluid (natural or synthetic). The seal geometry is intended to be representative and a variety of seal characteristics may be employed; such as a single lip seal, a double or multiple lip seal. A pad or wiper seal may be made from a variety of material options. Different seal mounting options may be used, for example a lobe in shaped groove as shown in FIG. 18, a retaining ring or clamp, adhesion substance. The seal may also be incorporated into the contact face of the interface zone.

The joint construction described above can be extended into a three-part configuration. For example, FIG. 20 illustrates a prosthetic joint 500 having first, second, and third members 502, 504, and 506. The first and second members 502 and 504 are similar in construction to the first member 102 described above, and each includes a body 508, an optional disk-like base 510, and a flange 512. The flanges 512 define wear-resistant concave first and second contact surfaces 514 and 516, each of which includes a protruding peripheral rim, and a recessed central portion as described above. The third member 506 has a double-convex shape defining opposed wear-resistant, convex third and fourth contact surfaces 524 and 526. The first and second 514 and 516 bear against the third and fourth contact surfaces 524 and 526, respectively, so as to transfer axial (i.e. compression) and lateral loads between the first and second members 502 and 504 through the third member 506, while allowing pivoting motion between the members 502, 504, and 506. The first and second contact surfaces 514 and 516 are conformal to the third and fourth contact surfaces 524 and 526 as described in more detail above.

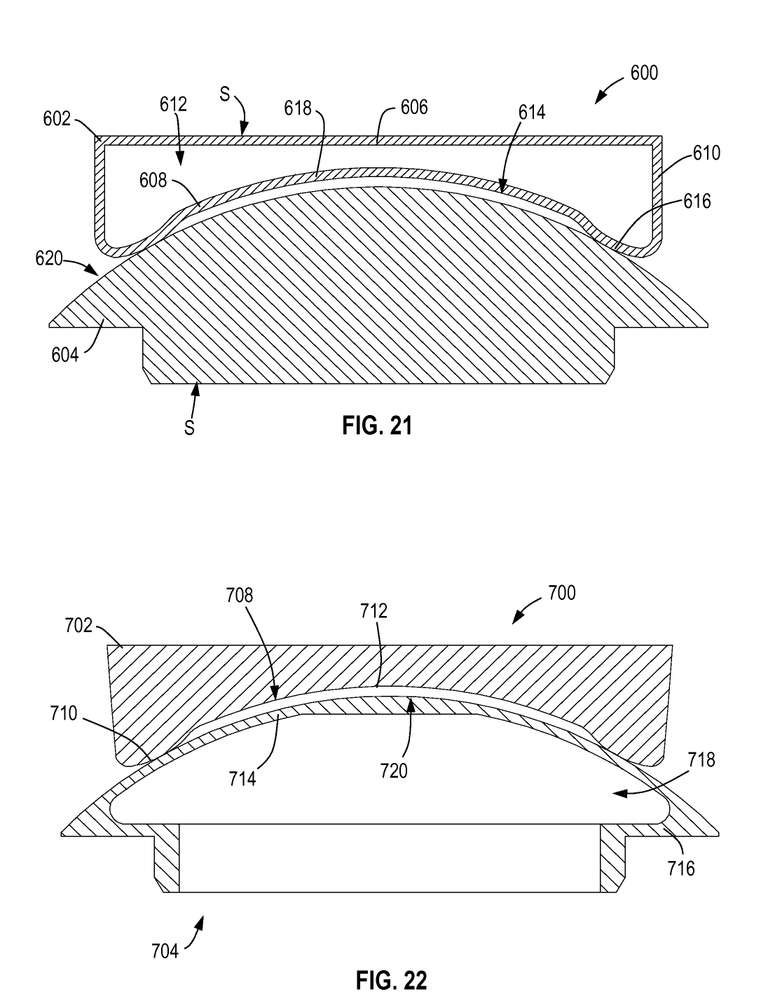

FIG. 21 illustrates an alternative prosthetic joint 600 comprising first and second members 602 and 604 constructed from rigid materials. Both of the members 602 and 604 may be bone-implantable, meaning they include osseointegration surfaces, labeled “S”, as described in more detail above.

The first member 602 is hollow and includes a disk-like base 606 and a cup 608, interconnected by a peripheral wall 610. An interior cavity 612 is defined between the base 606 and the cup 608. The cup 608 is constructed from a rigid material and defines a wear-resistant, concave first contact surface 614. The first contact surface 614 includes a protruding peripheral rim 616, and a recessed central portion 618, which may also be considered a “pocket” or a “relief”. The rim 616 may have a conical or curved cross-sectional shape. The interior cavity 612 may be filled with resilient nonmetallic material to provide damping and/or additional spring restoring force to the flange. Examples of suitable resilient materials include polymers, natural or synthetic rubbers, and the like.

The second member 604 is constructed from a rigid material and has a wear-resistant, convex second contact surface 620. The first and second contact surfaces 614 and 616 bear directly against each other so as to transfer axial and laterals loads from one member to the other while allowing pivoting motion between the two members 602 and 604.

As described above with reference to the prosthetic joint 100, the cup 606 of the first member 602 is thin enough to permit bending under working loads, but not so thin as to allow material yield or fatigue cracking. The first contact surface 614 is thus conformable to the second contact surface 620 when the prosthetic joint 600 is placed under external load.

An inverted configuration of hollow members is also possible. For example, FIG. 22 illustrates a prosthetic joint 700 comprising first and second members 702 and 704, both constructed of rigid materials. The first member 702 is solid and includes a wear-resistant, concave first contact surface 708. The first contact surface 708 includes a protruding peripheral rim 710, and a recessed central portion 712, which may also be considered a “pocket” or a “relief”.

The second member 704 is hollow and includes a dome 714 connected to a peripheral wall 716. An interior cavity 718 is defined behind the dome 714. The dome 714 defines a wear-resistant, convex second contact surface 720, which is shaped and sized enough to permit bending under working loads, but not so as to allow material yield or fatigue cracking The second contact surface 720 is thus conformable to the first contact surface 708 when the prosthetic joint 700 is placed under external load.

The first and second contact surfaces 708 and 720 bear directly against each other so as to transfer axial and lateral loads from one member to the other while allowing pivoting motion between the two members 702 and 704.

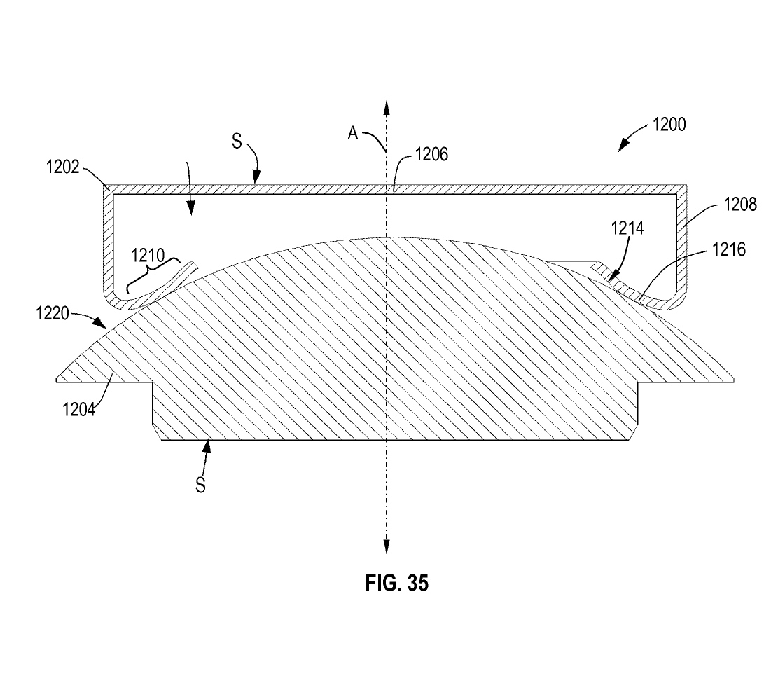

FIG. 35 illustrates another alternative prosthetic joint 1200 comprising first and second members 1202 and 1204 constructed from rigid materials. Both of the members 1202 and 1204 may be bone-implantable, meaning they may include osseointegration surfaces, labeled “S”, as described in more detail above.

The first member 1202 is hollow and includes a disk-like base 1206 with a peripheral wall 1208 extending axially from its edge. A flange 1210 extends from the distal end of the peripheral wall 1208, in a direction which is generally radial relative to a central axis A of the joint 1200. In this specific example it extends generally radially inward. The flange 1210 is constructed from a rigid material as described above and defines a wear-resistant, concave first contact surface 1214. The first contact surface 1214 includes a peripheral rim 1216 which protrudes relative to the remainder of the first contact surface 1214 (i.e. the remainder of the first contact surface 1214 may be considered to be “recessed” relative to the protruding peripheral rim 1216). The rim 1216 may have a conical or curved cross-sectional shape.

The second member 1204 is constructed from a rigid material and has a wear-resistant, convex second contact surface 1220. The first and second contact surfaces 1214 and 1216 bear directly against each other so as to transfer axial and laterals loads from one member to the other while allowing pivoting motion between the two members 1202 and 1204.

As described above with reference to the prosthetic joint 100, the flange 1210 (and thus the first contact surface 1214) of the first member 1202 is conformable to the second contact surface 1220 when the joint is placed under load. The flange 1210 is thin enough to permit bending under working loads, but not so thin as to allow material yield or fatigue cracking.

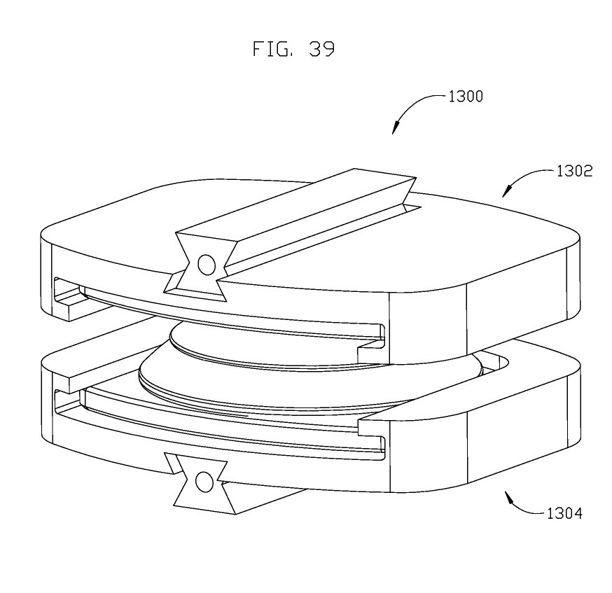

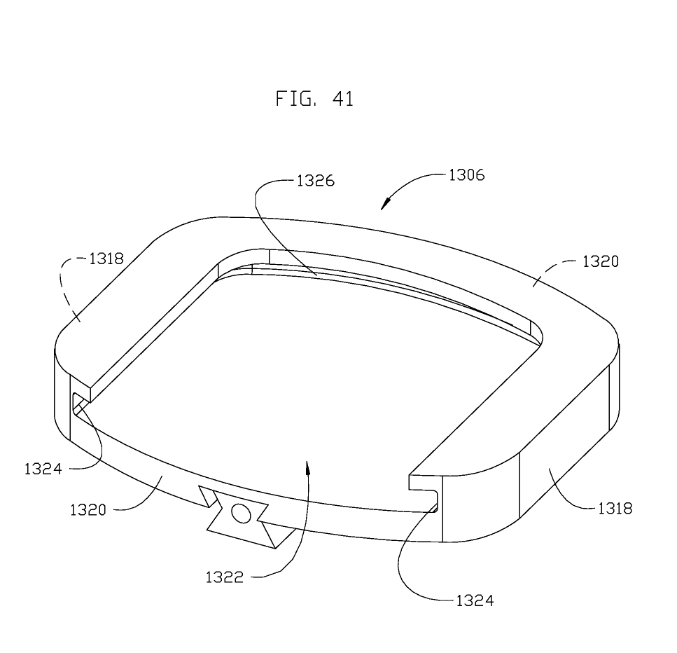

FIGS. 39-41 illustrate another alternative prosthetic joint 1300 comprising first and second members 1302 and 1304 constructed from rigid materials. The first member 1302 comprises a first or upper mounting member 1306 and a first or upper bearing member 1308. The second member 1304 comprises a second or lower mounting member 1310 and a second or lower bearing member 1312. As used herein, positional terms such as “upper” and “lower” are merely for convenient reference and do not imply any particular orientation of the elements described.

The first mounting member 1306 has an outer surface 1314 that is bone-implantable, meaning that it is configured to be infiltrated by bone growth to improve the connection between the implant and the bone. Bone implantable surfaces can have a coating (e.g. hydroxylapatite) or a non-smooth surface finish (textured, knurled, beaded, etc.) to promote bone integration. This type of surface is also sometimes referred to as an osseointegration surface as described above. In the illustrated example the outer surface 1314 is depicted as including a planar upper face 1316, opposed planar end faces 1318, and convex side faces 1320 extending between the end faces 1318, however it will be understood that various shapes could be substituted for a particular application.

Opposite to the outer surface 1314 the first mounting member 1306 includes a recessed receptacle 1322 defining a pair of spaced-apart parallel undercut side grooves 1324 interconnected by an undercut end groove 1326.

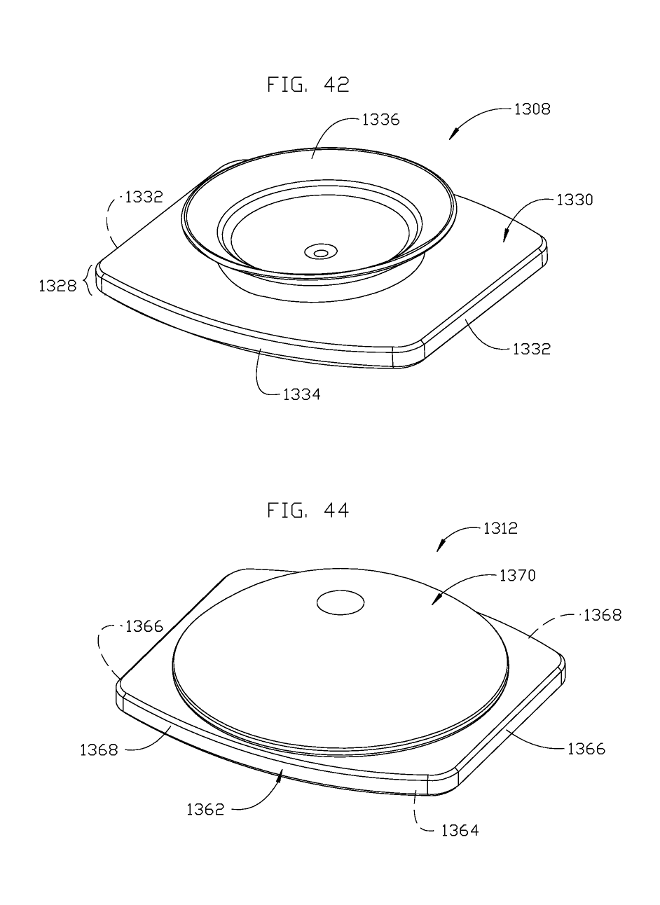

The first bearing member 1308 (see FIG. 42) is constructed from a rigid material and includes a base 1328 with a shape that is complementary to the receptacle 1322. In the illustrated example the base 1328 has a planar top surface 1330 bounded by two parallel, spaced-apart planar side edges 1332 and two convex end edges 1334. The base 1328 and the receptacle 1322 may be configured in size and shape to define a desired fit, for example of fit having a specified clearance between the two components or a specified interference between the two components, in order to facilitate installation and/or retention as desired. In this configuration, the first bearing member 1308 is removable from the first mounting member 1306. As used herein, the term “removable” implies the ability to remove the first bearing member 1308 from the first mounting member 1306 without resorting to damaging or destroying either component, i.e. without such processes as cutting, drilling, permanent bending, or the like. Generally the act of removing the first bearing member 1306 would involve sliding it out of the first mounting member 1306, possibly with the aid of a gripping or pulling tool.

As best seen in FIG. 43, the first bearing member 1308 includes a perimeter flange 1336 extending in a generally radially outward direction at one end. Without regard to the exact direction that the flange 1336 extends, a defining feature of the flange 1336 is that it is cantilevered relative to the base 1336. In other words, when viewed in cross-section, it is a projecting structure, that is supported at one end and carries a load at the other end or along its length. The flange 1336 may be open or closed perimeter, and may have varying shapes in plan view (e.g. circular, elliptical, a spline, or an asymmetrical shape). Optionally, the a portion of the base 1328 may define a protruding neck 1340 from which the flange 1336 may extend.

Collectively, the base 1328 and the flange 1336 define a wear-resistant, concave first contact surface 1342. The first contact surface 1342 includes a protruding peripheral rim 1344 and a recessed central portion 1346, which may also be considered a “pocket” or a “relief”. As used herein, the term “recessed” as applied to the central portion 1346 means that the central portion 1346 lies outside of the nominal exterior surface of the second bearing member 1312 when the joint 1300 is assembled. The terms “recessed” and “protruding” as used herein are opposite in meaning to one another. For example, the peripheral rim 1344 protrudes relative to a nominal surface defined by the central portion 1346, and the central portion 1346 is recessed relative to the rim 1344. The cross-sectional shape of the rim 1344 may be flat or curved as necessary to suit a particular application.

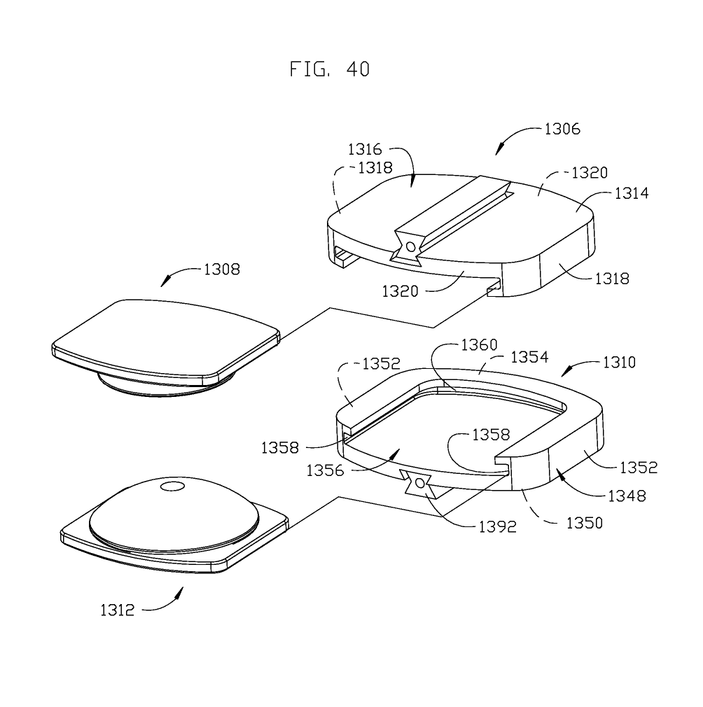

Referring back to FIG. 40, the second mounting member 1310 has an outer surface 1348 that is bone-implantable, meaning that it is configured to be infiltrated by bone growth to improve the connection between the implant and the bone. Bone implantable surfaces can have a coating or a non-smooth surface finish (textured, knurled, beaded, etc.) to promote bone integration. This type of surface is also sometimes referred to as an osseointegration surface as described above. In the illustrated example the outer surface 1348 is depicted as including a planar lower face 1350, opposed planar end faces 1352, and convex side faces 1354 extending between the end faces 1352, however it will be understood that various shapes could be substituted for a particular application.

Opposite to the outer surface 1348 the second mounting member 1310 includes a recessed receptacle 1356 defining a pair of spaced-apart parallel undercut side grooves 1358 interconnected by an undercut end groove 1360.

As seen in FIG. 44, the second bearing member 1312 includes a base 1362 with a shape that is complementary to the receptacle 1356. In the illustrated example the base 1362 has a planar bottom surface 1364 bounded by two parallel, spaced-apart planar side edges 1366 and two convex end edges 1368. The base 1362 and the receptacle 1356 may be configured in size and shape to define a desired fit, for example of fit having a specified clearance between the two components or a specified interference between the two components, in order to facilitate installation and/or retention as desired. In this configuration, the second bearing member 1312 is removable from the second mounting member 1310. As used herein, the term “removable” implies the ability to remove the second bearing member 1312 from the second mounting member 1310 without resorting to damaging or destroying either component, i.e. without such processes as cutting, drilling, permanent bending, or the like. Generally the act of removing the second bearing member 1312 would involve sliding it out of the second mounting member 1310, possibly with the aid of a gripping or pulling tool.

The second bearing member 1312 is constructed from a rigid material and has a wear-resistant, convex second contact surface 1370. The first and second contact surfaces 1342 and 1370 bear directly against each other so as to transfer axial and laterals loads from one member to the other while allowing pivoting motion between the two members 1302 and 1304.

As described above with reference to the prosthetic joint 100, the flange 1336 (and thus the first contact surface 1342) of the first member 1302 is conformable to the second contact surface 1370 when the joint 1300 is placed under load. The flange 1336 is thin enough to permit bending under working loads, but not so thin as to allow material yield or fatigue cracking.

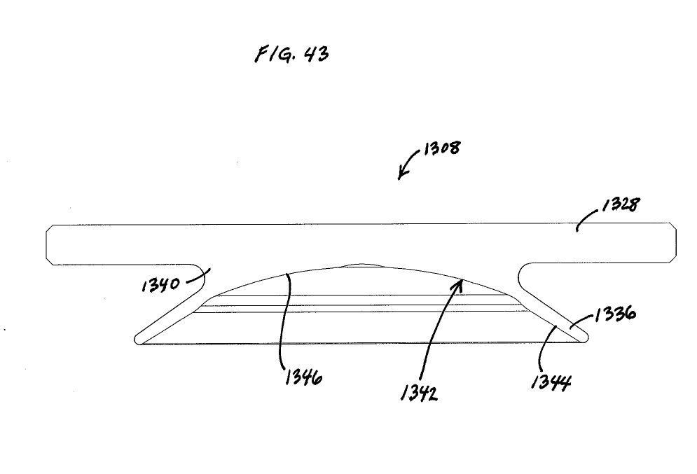

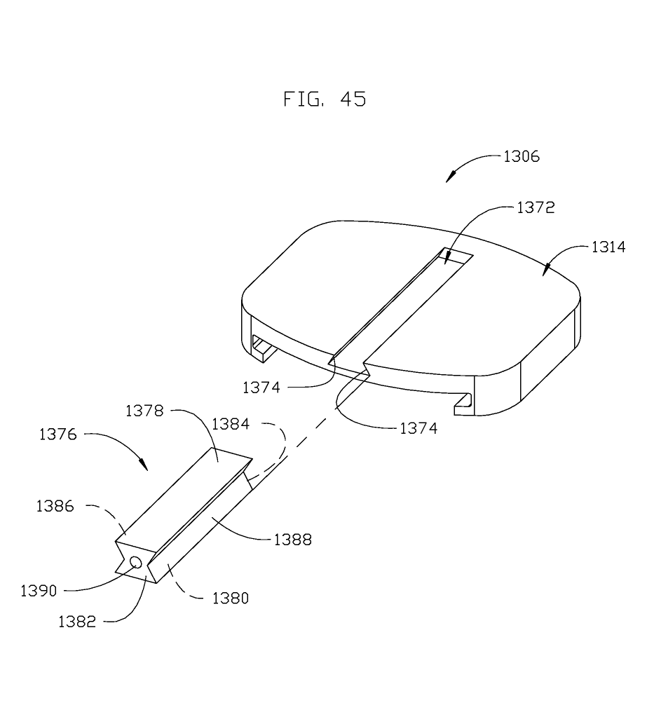

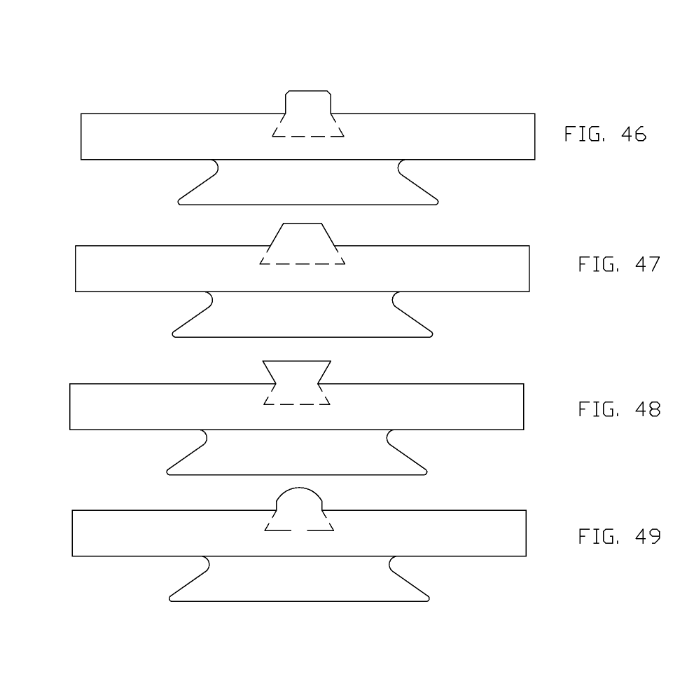

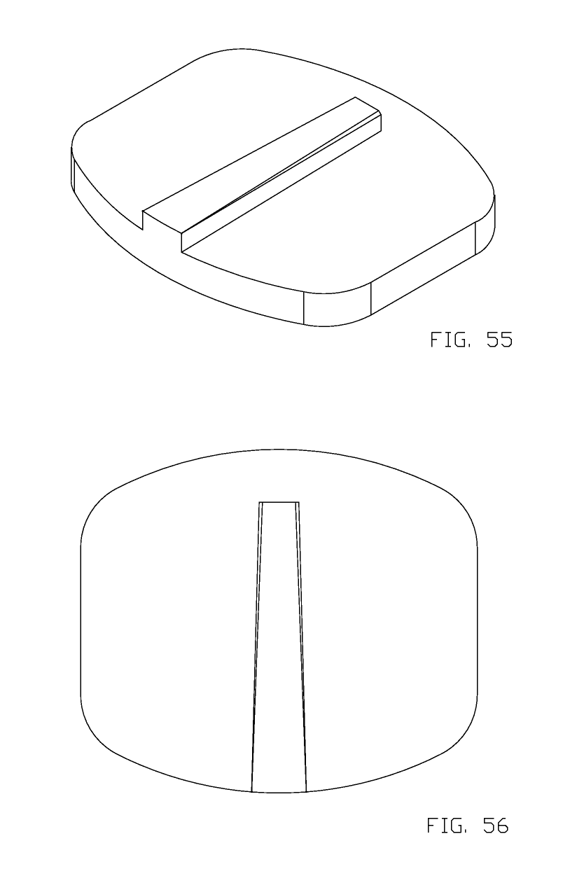

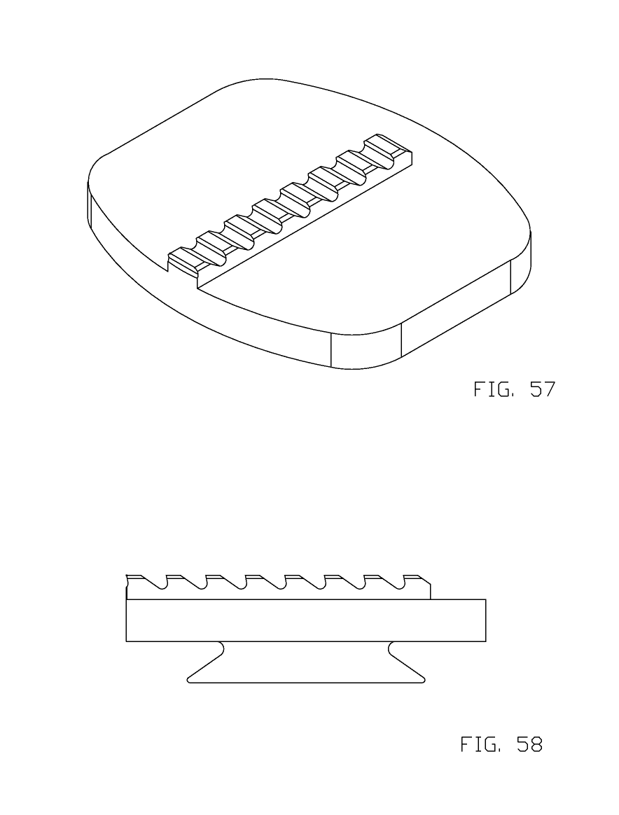

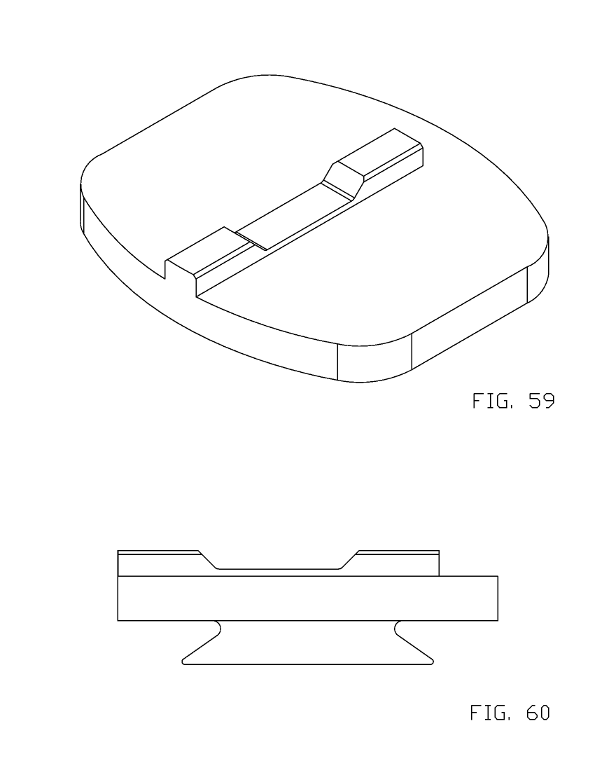

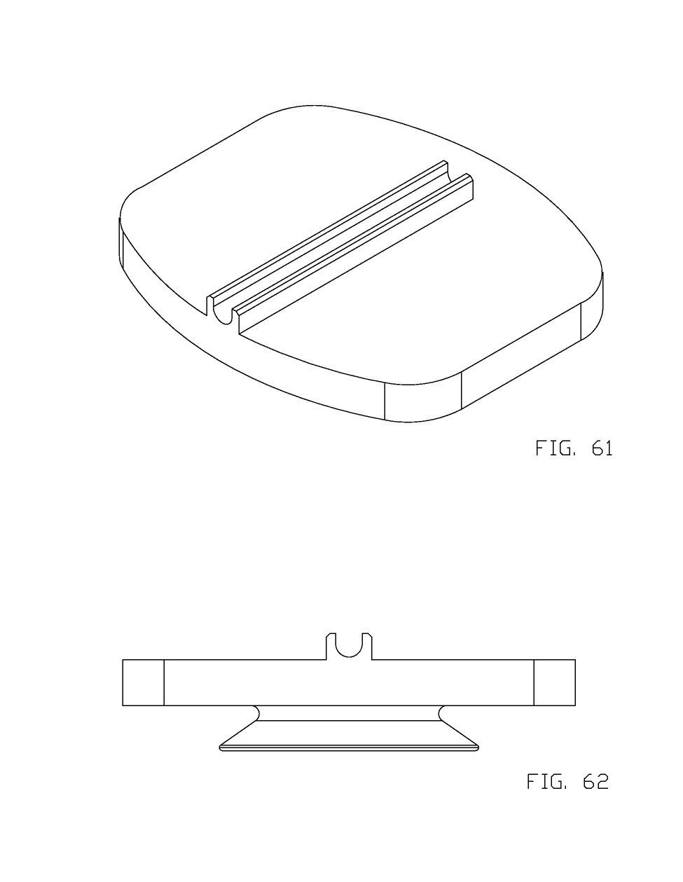

The joint 1300 may include at least one mechanical alignment feature configured to maintain the joint 1300 in a specific position and/or orientation relative to bone, either during or after implantation. For example, as shown in FIG. 45, the first mounting member 1306 includes a mechanical feature protruding from the outer surface 1314. In this particular example a slot 1372 with nonparallel side walls 1374 is formed in the outer surface 1314. An elongated key 1376 is received in the slot 1372. The key 1376 includes opposed top and bottom faces 1378 and 1380, opposed end faces 1382 and 1384, and opposed side faces 1386 and 1388. The portions of the side faces 1386 and 1388 that are received in the slot 1372 are shaped and sized to be complementary to the shape of the slot 1372. The protruding portion of the key 1376, that is, the portions of the side faces 1386 and 1388 that are exposed outside the outer surface 1314 and the top face 1378, may have various shapes. Examples of various shapes of the protruding portion (whether it be a key or a keel) are illustrated in FIGS. 46-49. In those figures, the dashed lines indicate the interface between a key (if present) and a surrounding groove. Further examples of shapes suitable for the protruding mechanical feature are illustrated in FIGS. 55-62. FIGS. 55 and 56 show a mechanical feature (representative of a keel or key) which is tapered in width along its long axis. FIGS. 57 and 58 show a mechanical feature having an upper surface interrupted with transverse cuts or grooves forming a series of lands or teeth. FIGS. 59 and 60 show a mechanical feature having an upper surface interrupted with a single elongated cut or groove dividing the mechanical feature into two separate lands. FIGS. 61 and 62 show a mechanical feature having an upper surface with a single elongated cut or groove formed therein. The groove has a radiused bottom and runs parallel to the long axis of the mechanical feature.

The key 1372 may incorporate a feature for engaging an instrument used to insert, manipulate, and/or remove the key 1372 or the member attached to the key 1372. In the illustrated example this is implemented as a threaded hole 1390 in the end face 1382 of the key 1372.

Alternatively, the alignment feature may be implemented as a “keel” having an incorporated as an integral part of the first mounting member 1306. FIGS. 46-49 illustrate various exterior shapes which may be defined by a key or keel structure. In FIGS. 46-49, the solid lines indicate the exterior profile of the key or keel, while the dashed lines indicate the intersection between the key and the slot, if a key is used.

The second member 1304 may also incorporate an alignment feature such as a key or keel, which is the same as or different from the alignment feature of the first member 1302. In the example shown in FIGS. 39 and 40 the second member 1304 includes a key 1392.

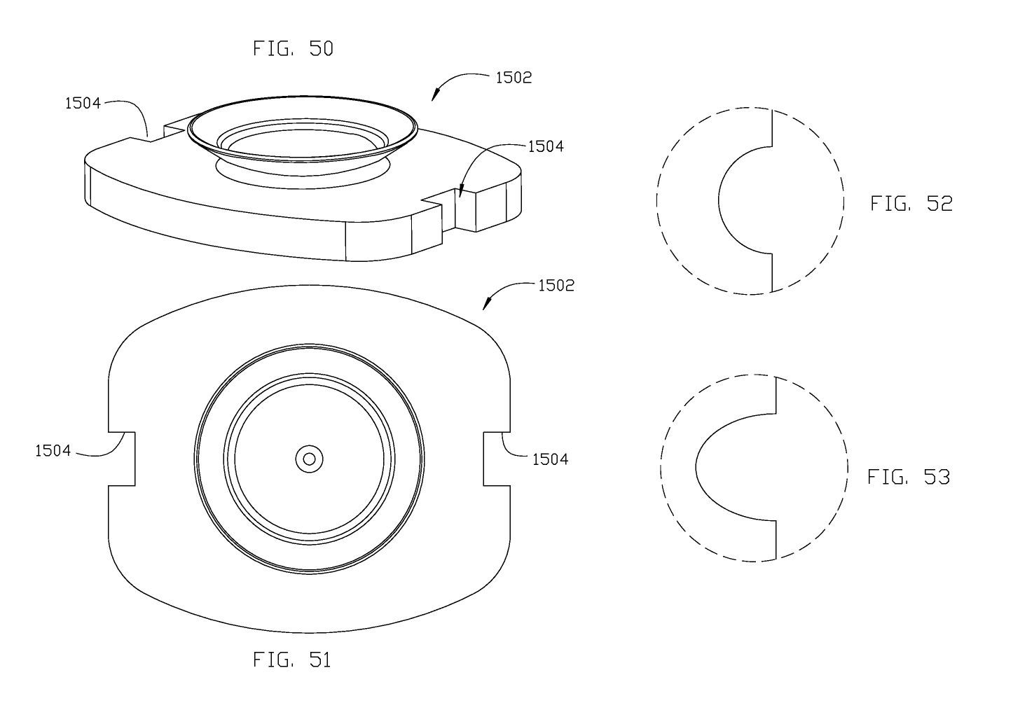

Any of the joint members described above may incorporate one or more features for engaging an instrument for installation, positioning or alignment. For example, FIGS. 50 and 51 illustrate a joint member 1502 having notches 1504 formed therein. The notches may have varying shapes, as seen in FIGS. 52 and 53.

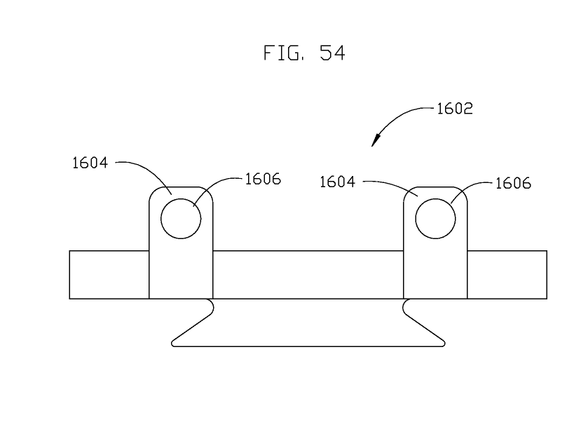

Any of the joint members described above may incorporate one or more features for securing the joint member to bone. For example, FIG. 54 illustrates a joint member 1602 having tabs 1604 extending therefrom. The tabs 1604 include holes 1606 through which screws or other fasteners can be driven into bone.

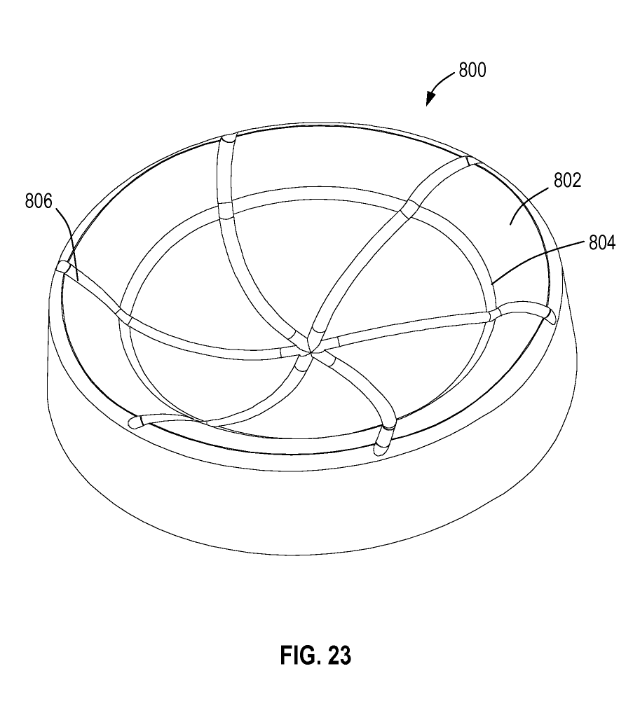

Any of the contact surfaces described above may be provided with one or more grooves formed therein to facilitate flow of fluid or debris. For example, FIG. 23 illustrates a joint member 800 including a concave contact surface 802. The contact surface 802 includes a circular groove 804, and plurality of generally radially-extending grooves 806 which terminate at the center of the contact surface 802 and intersect the circular groove 804.

As noted above, known coatings such as titanium nitride, chrome plating, carbon thin films, and/or diamond-like carbon coatings may be used to impart wear resistance or augment the wear resistance of any of the first contact surfaces described above. To the same end, it may be desirable to surface treat either or both interfaces of any of the above-described implants or joints with a laser, shot peen, burnishing, or water shock process, to impart residual compressive stresses and reduce wear. The benefit could be as much from surface annealing and microstructure and microfracture elimination as smoothing itself.

The foregoing has described medical implants and prosthetic joints with wear-resistant properties and conformal geometries. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

What is claimed is:

1. A prosthetic joint, comprising:
(a) a first member including a bone-implantable surface, the first member comprising a rigid material and including a body having a cantilevered perimeter flange extending therefrom, the flange defining a wear-resistant, concave first contact surface having a protruding rim and a recessed central portion; and
(b) a second member including a bone-implantable surface, the second member comprising a rigid material with a wear-resistant, convex second contact surface;
(c) where the first and second contact surfaces bear directly against each other so as to transfer axial and lateral loads from one of the members to the other, while allowing pivoting motion between the two members;
(d) wherein the flange is shaped and sized so as to deform elastically and permit the first contact surface to conform in an irregular shape to the second contact surface when the joint is placed under a predetermined load; and
(e) wherein at least one of the bone-implantable surfaces includes at least one mechanical feature configured to maintain the associated member in a specific position and/or orientation relative to bone, either during or after implantation.

2. The prosthetic joint of claim 1, wherein at least one of the first and second members is of two-piece construction including a mounting member defining the bone-implantable surface and a bearing member defining the contact surface, wherein the bearing member is removable from the mounting member.

3. The prosthetic joint of claim 1 wherein the mechanical feature is an elongated protruding keel configured to be received in a groove machined in a bone.

4. The prosthetic joint of claim 1 wherein the mechanical feature is a key received in a slot in the bone-implantable surface and defining an elongated protruding feature configured to be received in a groove machined in a bone.

5. The prosthetic joint of claim 1, wherein the first contact surface has a radiused cross-sectional shape.

6. The prosthetic joint of claim 1, wherein the first contact surface has a conical cross-sectional shape.

7. The prosthetic joint of claim 1, wherein the contact surfaces are ceramic, metallic, or a combination thereof.

8. The prosthetic joint of claim 1, where the flange is sized so as to permit elastic deflection of the flange while limiting stresses in the flanges to less than the endurance limit of the material, when a predetermined external axial load is applied to the joint.

9. The prosthetic joint of claim 1 where the curvatures of the first and second contact surfaces are configured to produce an asymptotic wear characteristic when in use.

10. The prosthetic joint of claim 1 where the bone-implantable surface includes a coating that promotes bone growth.

11. The prosthetic joint of claim 1 where the bone-implantable surface includes a surface texture that promotes bone growth.

12. The prosthetic joint of claim 1 wherein at least one of the joint members includes at least one notch configured to engage an instrument for manipulation of the joint.

13. The prosthetic joint of claim 1 wherein at least one of the joint members includes a bracket configured for face-screwing the implant to bone.
